(19) United States
(12) Patent Application Publication
(10) Pub. No.: US 2022/0260609 A1
(43) Pub. Date: Aug. 18, 2022

(54) DETERMINATION DEVICE, DETERMINATION METHOD, AND PROGRAM RECORDING MEDIUM

(71) Applicant: NEC Corporation, Minato-ku, Tokyo (JP)

(72) Inventors: Chenhui HUANG, Tokyo (JP); Kazuki IHARA, Tokyo (JP); Noriyuki TONOUCHI, Tokyo (JP); Kenichiro FUKUSHI, Tokyo (JP)

(73) Assignee: NEC Corporation, Minato-ku, Tokyo (JP)

(21) Appl. No.: 17/607,924

(22) PCT Filed: May 15, 2019
(86) PCT No.: PCT/JP2019/019211

§ 371 Date: Nov. 1, 2021

(51) Int. Cl. G01P 15/08; G01P 15/18

(52) U.S. Cl. CPC G01P 15/08; G01P 15/18

(57) ABSTRACT

A determination device includes: a sensor data reception unit that receives sensor data including acceleration in a travel direction of a user and in a gravity direction; a walking state discrimination unit that switches to a discrimination mode in response to the acceleration in the gravity direction exceeding a first threshold in a power saving mode, and switches to a walk measurement mode in response to the acceleration in the travel direction exceeding a second threshold in the discrimination mode; a change trend detection unit that detects a change trend of peak values using log data of the peak values of the acceleration in the travel direction in the discrimination mode; a threshold change unit that changes the second threshold based on a detection result by the change trend detection unit; and a transmission unit that transmits the sensor data in the walk measurement mode.

### TECHNICAL FIELD

The present invention relates to a determination device, a determination method, and a program for determining a walking state.

### BACKGROUND ART

In response to growing interest in healthcare for physical condition management, a technology of measuring a walk using sensor data acquired by a sensor attached to a body has been developed. In order to stably perform walk measurement for a long time, power saving of a sensor that acquires sensor data and a device that determines or measures a walk using the sensor data is required.

PTL 1 discloses an electronic device that determines an action mode of a user based on at least one of an angular velocity signal output from an angular velocity sensor and an acceleration signal output from an acceleration sensor. The electronic device of PTL 1 reduces power supplied to the angular velocity sensor in a case of determining that the user starts walking based on the angular velocity signal or both the angular velocity signal and the acceleration signal. That is, the electronic device of PTL 1 shifts the angular velocity sensor to a power saving mode to reduce power consumption when the user starts walking, and automatically shifts the angular velocity sensor from the power saving mode to a normal mode when the user stops walking.

PTL 2 discloses a mobile terminal device that wirelessly operates an operation target. The device of PTL 2 stores in advance a table in which time information is associated with a threshold. When acquiring the time information, the device of PTL 2 refers to the table and selects the threshold associated with the time information. The device of PTL 2 then determines whether to cancel the power saving mode based on a comparison result between the selected threshold and the number of steps of the user who operates the mobile terminal device.

### CITATION LIST

### Patent Literature

[PTL 1] WO 2014/122903 A
[PTL 2] JP 2014-168113 A

### SUMMARY OF INVENTION

### Technical Problem

In the method of PTL 1, a threshold set in advance for acceleration, angular velocity and the like is used as a determination criterion for mode shift. Therefore, in the method of PTL 1, there is a case where mode switching is not performed flexibly, for example, at an appropriate timing, in accordance with a walking state of the user that changes according to a fatigue level and the like.

In the method of PTL 2, two thresholds (high and low) are stored for the number of steps in association with the time information, and it shifts to or cancels the power saving mode based on the comparison result between the threshold related to current time information and the number of steps. In the method of PTL 2, power saving is achieved by making it difficult to cancel the power saving mode in a case where it is highly possible that an operation on the operation target is not intended. In the method of PTL 2, since the two thresholds (high and low) are fixed values, there is a case where the power saving mode is hardly canceled in a case where the walking state of the user fluctuates.

An object of the present invention is to solve the above-described problems and provide a determination device and the like that achieves high efficiency and low power consumption of walk measurement while flexibly responding to a change in walking state.

### Solution to Problem

A determination device according to an aspect of the present invention is provided with a sensor data reception unit for receiving sensor data including acceleration in a travel direction of a user and in a gravity direction, a walking state discrimination unit for switching to a discrimination mode when the acceleration in the gravity direction exceeds a first threshold in a power saving mode, and switching to a walk measurement mode when the acceleration in the travel direction exceeds a second threshold in the discrimination mode, a change trend detection unit for detecting a change trend of peak values using log data of the peak values of the acceleration in the travel direction in the discrimination mode, a threshold change unit for changing the second threshold based on a detection result by the change trend detection unit, and a transmission unit for transmitting the sensor data in the walk measurement mode.

In a determination method according to an aspect of the present invention, sensor data including acceleration in a travel direction of a user and in a gravity direction is received, it is switched to a discrimination mode when the acceleration in the gravity direction exceeds a first threshold in a power saving mode, it is switched to a walk measurement mode when the acceleration in the travel direction exceeds a second threshold in the discrimination mode, the sensor data is transmitted in the walk measurement mode, a change trend of peak values is detected using log data of the peak values of the acceleration in the travel direction in the discrimination mode, and the second threshold is changed based on the change trend of the peak values.

A program according to an aspect of the present invention causes a computer to execute processing for receiving sensor data including to acceleration in a travel direction of a user and in a gravity direction, processing for switching to a discrimination mode when the acceleration in the gravity direction exceeds a first threshold in a power saving mode, processing for switching to a walk measurement mode when the acceleration in the travel direction exceeds a second threshold in the discrimination mode, processing for transmitting the sensor data in the walk measurement mode, processing for detecting a change trend of peak values using log data of the peak values of the acceleration in the travel direction in the discrimination mode, and processing for changing the second threshold based on the change trend of the peak values.

### Advantageous Effects of Invention

According to the present invention, it becomes possible to provide a determination device and the like that achieves high efficiency and low power consumption of walk measurement while flexibly responding to a change in walking state.

### BRIEF DESCRIPTION OF DRAWINGS

FIG. 1 is a block diagram illustrating an example of a configuration of a walk measurement system according to a first example embodiment of the present invention.

FIG. 2 is a conceptual diagram illustrating an arrangement example of a walk determination device according to the first example embodiment of the present invention.

FIG. 3 is a conceptual diagram for illustrating a coordinate system of sensor data acquired by a data acquisition device according to the first example embodiment of the present invention.

FIG. 4 is a conceptual diagram for illustrating a gait cycle measured by the walk measurement device according to the first example embodiment of the present invention.

FIG. 5 is a block diagram illustrating an example of a configuration of a determination device according to the first example embodiment of the present invention.

FIG. 6 is a graph illustrating an example of a waveform regarding a change in time of acceleration in a travel direction acquired by the determination device according to the first example embodiment of the present invention.

FIG. 7 is a block diagram illustrating an example of a configuration of the data acquisition device according to the first example embodiment of the present invention.

FIG. 8 is a flowchart illustrating an example of an operation in mode switching by the determination device according to the first example embodiment of the present invention.

FIG. 9 is a flowchart illustrating an example of an operation of a change trend determination unit of the determination device according to the first example embodiment of the present invention.

FIG. 10 is a conceptual diagram illustrating an example of a configuration of a determination device according to a second example embodiment of the present invention.

FIG. 11 is a graph illustrating an example of a relationship between a step count used by the determination device according to the second example embodiment of the present invention to discriminate a walking state and an extreme value of acceleration in a travel direction.

FIG. 12 is a graph illustrating another example of a relationship between a step count used by the determination device according to the second example embodiment of the present invention to discriminate a walking state and acceleration in a travel direction.

FIG. 13 is a flowchart illustrating an example of an operation of a change trend determination unit of the determination device according to the second example embodiment of the present invention.

FIG. 14 is a conceptual diagram illustrating an example of a configuration of a determination device according to a third example embodiment of the present invention.

FIG. 15 is a graph illustrating an example of a relationship between a step count used by the determination device according to the third example embodiment of the present invention to discriminate a walking state and acceleration in a travel direction.

FIG. 16 is a flowchart illustrating an example of an operation of a change trend determination unit of the determination device according to the third example embodiment of the present invention.

FIG. 17 is a conceptual diagram illustrating an example of a configuration of a determination device according to a fourth example embodiment of the present invention.

FIG. 18 is a flowchart illustrating an example of an operation of a change trend determination unit of the determination device according to the fourth example embodiment of the present invention.

FIG. 19 is a conceptual diagram illustrating an example of a configuration of a determination device according to a fifth example embodiment of the present invention.

FIG. 20 is a conceptual diagram illustrating an example of displaying notification information according to a walking state on a display unit of a mobile terminal in which a walk measurement device according to the fifth example embodiment of the present invention is implemented.

FIG. 21 is a conceptual diagram illustrating another example of displaying notification information according to a walking state on a display unit of a mobile terminal in which a walk measurement device according to the fifth example embodiment of the present invention is implemented.

FIG. 22 is a flowchart illustrating an example of an operation of a change trend determination unit of the determination device according to the fifth example embodiment of the present invention.

FIG. 23 is a conceptual diagram illustrating a hardware configuration for achieving the walk measurement system according to each example embodiment of the present invention.

### EXAMPLE EMBODIMENT

Hereinafter, example embodiments of the present invention are described with reference to the drawings. Note that, the example embodiments described below have technically preferable limitations for carrying out the present invention, but the scope of the invention is not limited to the following. In all the drawings used in the following description of the example embodiment, the same reference numeral is given to the same parts unless there is a particular reason. In the following example embodiments, repeated description of similar configurations and operations are sometimes omitted. A direction of an arrow in the drawings is an example, and does not limit a direction of a signal between blocks.

### First Example Embodiment

First, a walk measurement system according to a first example embodiment of the present invention is described with reference to the drawings. The walk measurement system of the present example embodiment calculates an attitude angle using sensor data acquired by an acceleration sensor and an angular velocity sensor arranged in footwear such as shoes, and measures a walk based on time-series data of the attitude angle. For example, the walk measurement system of the present example embodiment performs walk measurement by calculating the attitude angle using acceleration data and angular velocity data acquired by an inertial measurement unit (IMU) arranged in a sole insert (also referred to as an insole). The walk measurement system of the present example embodiment switches an operation mode using the sensor data using the acceleration data acquired by the acceleration sensor.

(Configuration)

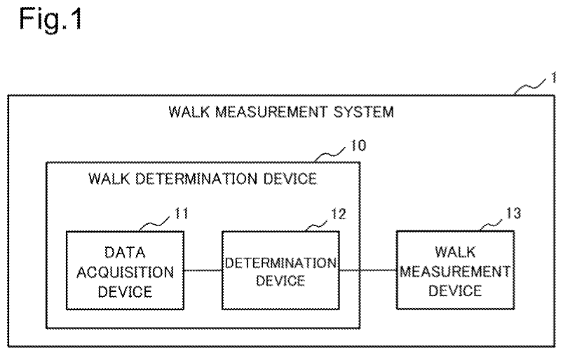

FIG. 1 is a block diagram illustrating an example of a configuration of a walk measurement system 1 of the present example embodiment. The walk measurement system 1 is provided with a data acquisition device 11, a determination device 12, and a walk measurement device 13. The data acquisition device 11 and the determination device 12 form a walk determination device 10. The data acquisition device 11 and the determination device 12 may be connected to each other by wire or wirelessly. The determination device 12 and the walk measurement device 13 may be connected to each other by wire or wirelessly. A display device (not illustrated) that displays a determination result of the determination device 12 and a measurement result of the walk measurement device 13 may also be connected to the walk measurement device 13.

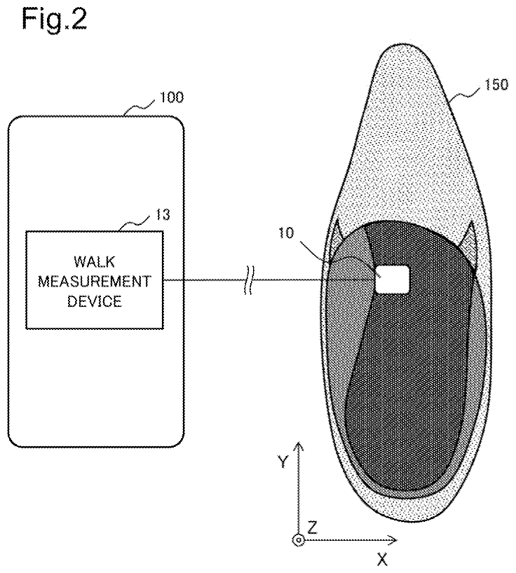

FIG. 2 is a conceptual diagram illustrating an example in which the walk determination device 10 is arranged in a shoe 150. In the example in FIG. 2, the walk determination device 10 is arranged in a position below the arch of the foot. The position where the walk determination device 10 is installed may also be a position other than the position of the arch of the foot as long as the position is inside or on a surface of the shoe 150. For example, the walk determination device 10 may be arranged in the insole inserted into the shoe 150. The walk determination device 10 may also be arranged in footwear other than shoes, socks and the like as long as this may determine a walking state.

In the example in FIG. 2, the walk measurement device 13 is configured in a mobile terminal 100 such as a smartphone and a tablet carried by a user. The walk measurement device 13 may be an application installed in the mobile terminal 100 or may be a dedicated circuit. The walk measurement device 13 may also be a logic circuit configured in a reconfigurable field-programmable gate array (FPGA). For example, a display unit (not illustrated) of the mobile terminal 100 is allowed to display the determination result of the determination device 12 and the measurement result of the walk measurement device 13.

As illustrated in FIG. 1, the data acquisition device 11 is connected to the determination device 12. The data acquisition device 11 at least includes an acceleration sensor and an angular velocity sensor. The data acquisition device 11 converts data acquired by the acceleration sensor and the angular velocity sensor into digital data (also referred to as sensor data), and transmits the converted sensor data to the determination device 12. The data acquisition device 11 and the walk measurement device 13 may also be directly connected to each other without the determination device 12.

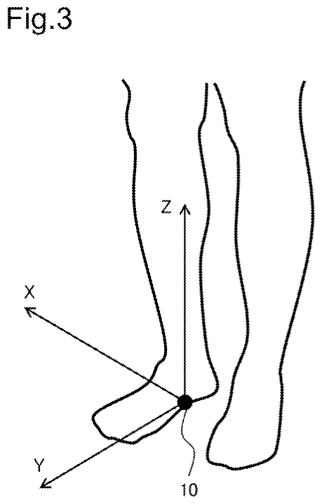

FIG. 3 is a conceptual diagram for illustrating a coordinate system of the sensor data acquired by the data acquisition device 11. In the example in FIG. 3, a lateral direction of a walker is set to an X direction (positive in a rightward direction), a travel direction of the walker is set to a Y direction (positive in a forward direction), and a gravity direction is set to a Z direction (positive in a vertically upward direction). Hereinafter, unless otherwise specified, the lateral direction, the travel direction, and the gravity direction of the walker indicate the X direction, the Y direction, and the Z direction in FIG. 3, respectively.

The data acquisition device 11 is achieved by, for example, an inertial measurement device including the acceleration sensor and the angular velocity sensor. An example of the inertial measurement device is an IMU. The IMU includes a triaxial acceleration sensor and a triaxial angular velocity sensor. An example of the inertial measurement device is a vertical gyro (VG). The VG has a configuration similar to that of the IMU, and may output a roll angle and a pitch angle with reference to the gravity direction by a method referred to as strap down. An example of the inertial measurement device is an attitude heading reference system (AHRS). The AHRS has a configuration obtained by adding an electronic compass to the VG. The AHRS may output a yaw angle in addition to the roll angle and the pitch angle. An example of the inertial measurement device is a global positioning system/inertial navigation system (GPS/INS). The GPS/INS has a configuration obtained by adding a GPS to the AHRS. Since the GPS/INS may calculate a position in a three-dimensional space in addition to the attitude angle (roll angle, pitch angle, and yaw angle), this may estimate the position with high accuracy.

In a case where the acceleration data is used, the attitude angle may be calculated from magnitude of acceleration applied in an axial direction of each of a pitch axis and a roll axis. In a case where the angular velocity data is used, by integrating values of the angular velocities around the pitch axis, the roll axis, and the yaw axis as central axes, it is possible to calculate the attitude angle around the axes. High-frequency noise changing in various directions is included in the acceleration data, and low-frequency noise always in the same direction is included in the angular velocity data. Therefore, by removing a high-frequency component by applying a low-pass filter to the acceleration data, removing a low-frequency component by applying a high-pass filter to the angular velocity data, and combining outputs thereof, it is possible to improve accuracy of the sensor data from a foot portion in which noise is easily included. By applying a complementary filter to each of the acceleration data and the angular velocity data and taking a weighted average thereof, the accuracy of the sensor data may be improved.

As illustrated in FIG. 1, the determination device 12 is connected to the data acquisition device 11 and the walk measurement device 13. The determination device 12 acquires the sensor data from the data acquisition device 11. For example, the determination device 12 is achieved by a microcomputer.

The determination device 12 switches an operation mode of the walk measurement system 1 according to a value of the acceleration data included in the sensor data from the data acquisition device 11. The walk measurement system 1 operates in at least three operation modes including a power saving mode, a discrimination mode, and a walk measurement mode. For example, in order to reduce power consumption, acquisition frequency and discrimination frequency of the data are decreased in the power saving mode as compared with the discrimination mode and the walk measurement mode.

The power saving mode is the mode in which the data acquisition device 11 generates the sensor data including at least the acceleration data by a low-speed operation, and the determination device 12 determines the walking state using the acceleration in the gravity direction (Z direction). The low-speed operation in the power saving mode is the operation in which the sensor data is generated at a lower speed than a generation processing speed in a case where the sensor data is generated in the discrimination mode and the measurement mode. The discrimination mode is the mode in which the data acquisition device 11 generates the sensor data including at least the acceleration data by a high-speed operation, and the determination device 12 determines the walking state using the acceleration in the travel direction (Y direction). The walk measurement mode is the mode in which the data acquisition device 11 generates the sensor data including the acceleration data and the angular velocity data by the high-speed operation, and the walk measurement device 13 performs walk measurement based on the determination result by the determination device 12. It is also possible to configure such that the discrimination mode and the walk measurement mode operate in parallel. It is possible to configure such that, in the power saving mode, the walk measurement device 13 is set to a standby state of low power consumption. It is only required that the determination result be information that indicates or discriminates the walking state.

The determination device 12 switches the operation mode using two thresholds (first threshold and second threshold). The first threshold is the threshold set for the acceleration in the gravity direction (Z direction) in order to switch the operation mode from the power saving mode to the discrimination mode. The second threshold is the threshold set for the acceleration in the travel direction (Y direction) after it is switched from the power saving mode to the discrimination mode.

In the power saving mode, when the acceleration in the gravity direction (Z direction) exceeds the first threshold, the determination device 12 switches from the power saving mode to the discrimination mode. When switching from the power saving mode to the discrimination mode, the determination device 12 stores a log of peak values of time-series data of the acceleration in the travel direction (Y direction).

In the discrimination mode, the determination device 12 discriminates the walking state using the acceleration in the travel direction (Y direction). In a case where the acceleration in the travel direction (Y direction) exceeds the second threshold under a certain condition, the determination device 12 determines that a walk is started. When determining that the walk is started, the determination device 12 switches from the discrimination mode to the walk measurement mode and transmits the sensor data including the acceleration data and the angular velocity data to the walk measurement device 13.

In the discrimination mode, when the log of the acceleration in the travel direction (Y direction) of a predetermined number of steps (for example, 10 steps) is accumulated, the determination device 12 reads the log of the acceleration in the travel direction (Y direction) of the predetermined number of steps. The determination device 12 determines a change trend of the walking state using a waveform of the time-series data of the acceleration in the travel direction (Y direction) (also referred to as a walking waveform). For example, the determination device 12 determines the change trend of the walking state based on a change in extreme value (also referred to as peak value) such as a maximum value or a minimum value of the walking waveform. The determination device 12 resets the second threshold according to a determination result of the change trend of the walking state. For example, the change trend determination unit 133 extracts peaks of the 10 steps from the most recently measured walking waveform, detects a change trend of the extracted peak values, and resets the second threshold based on the detected change trend.

The walk measurement device 13 receives the sensor data from the data acquisition device 11 via the determination device 12. The walk measurement device 13 that receives the sensor data from the determination device 12 starts the walk measurement using the sensor data as a trigger. The configuration and operation of the walk measurement device 13 are not especially limited as long as the walk measurement is executed using the sensor data. It is also possible to configure such that the walk measurement device 13 directly receives a sensor signal from the data acquisition device 11 in response to a trigger signal output when the determination device 12 shifts to the walk measurement mode.

For example, the walk measurement device 13 calculates the attitude angle using the received sensor data. In the present example embodiment, the attitude angle is an angle of a sole surface with respect to a horizontal plane (ground). The walk measurement device 13 generates the time-series data of the attitude angle. The walk measurement device 13 generates the time-series data of the attitude angle with a general gait cycle or at a predetermined timing or a predetermined time interval set in accordance with a gait cycle unique to the user. The walk measurement device 13 executes the walk measurement using the time-series data of the attitude angle. The walk measurement device 13 outputs analysis of a walking phase and measurement results of a stride length, a walking speed, a sensor height and the like to a display device (not illustrated) and another system.

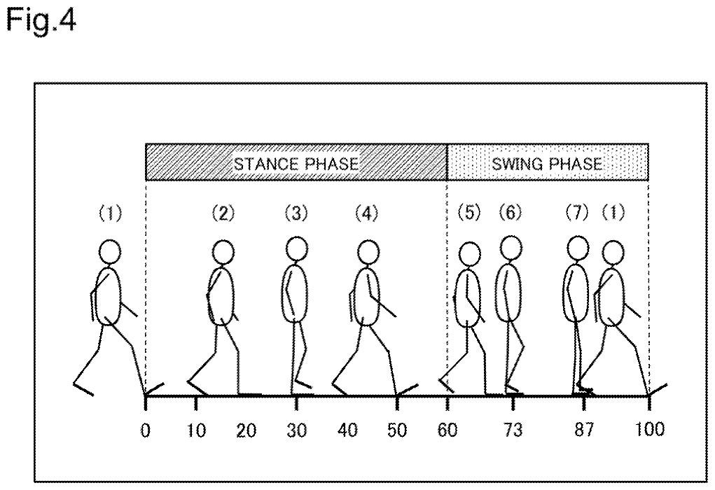

FIG. 4 is a conceptual diagram for illustrating the gait cycle measured by the walk measurement device 13. Time normalized with one gait cycle of one leg as 100% (also referred to as normalized time) is plotted along the abscissa in FIG. 4. In general, one gait cycle of one foot is broadly divided into a stance phase in which at least a part of the sole of the foot is in contact with the ground and a swing phase in which the sole of the foot is away from the ground. In the example in FIG. 4, the stance phase and the swing phase occupy about 60% and 40%, respectively, of one gait cycle. In the example in FIG. 4, events such as initial contact (1), contralateral toe contact (2), heel lifting (3), contralateral initial contact (4), toe contact (5), both feet proximity (6), and vertical tibia (7) appear in one gait cycle. The walk measurement system 1 detects a pattern caused by these events from a change in time of the attitude angle, and performs the walk measurement based on the detected pattern.

For example, the walk measurement device 13 is achieved by software (application) installed in a mobile terminal such as a smartphone, a mobile phone, a tablet, or a notebook personal computer or a circuit. For example, in a case where the walk measurement device 13 is used for data analysis of research and the like, this may be achieved by software installed in an information processing device such as a stationary computer or a server or a circuit.

The configuration of the walk measurement system 1 is described above. The configuration of the walk measurement system 1 in FIG. 1 is an example, and the configuration of the walk measurement system 1 of the present example embodiment is not limited to this form.

[Determination Device]

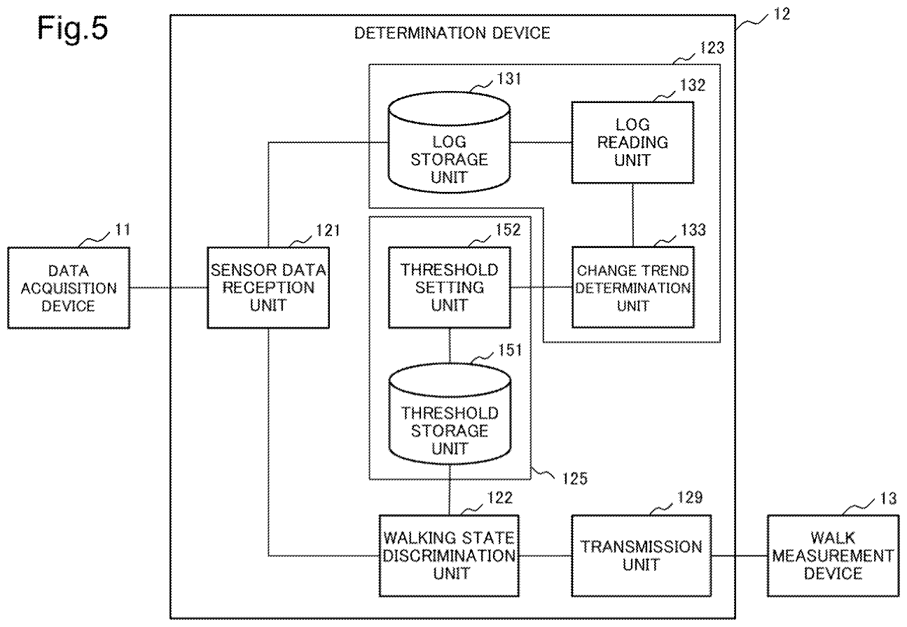

Next, a configuration of the determination device 12 is described with reference to the drawings. FIG. 5 is a block diagram illustrating an example of the configuration of the determination device 12. As illustrated in FIG. 5, the determination device 12 includes a sensor data reception unit 121, a walking state discrimination unit 122, a change trend detection unit 123, a threshold change unit 125, and a transmission unit 129. As an example, the change trend detection unit 123 in FIG. 5 includes a log storage unit 131, a log reading unit 132, and a change trend determination unit 133. As an example, the threshold change unit 125 in FIG. 5 includes a threshold storage unit 151 and a threshold setting unit 152. For example, each component of the determination device 12 may be achieved by a microcomputer including a dedicated circuit, or may be achieved by software implemented in the microcomputer. Each component of the determination device 12 may be achieved by a logic circuit configured in a reconfigurable FPGA.

The sensor data reception unit 121 is connected to the data acquisition device 11, the walking state discrimination unit 122, and the log storage unit 131. Although not illustrated in FIG. 5, the sensor data reception unit 121 and the log reading unit 132 may be connected to each other by a signal line (not illustrated). The sensor data reception unit 121 receives the sensor data from the data acquisition device 11. The sensor data reception unit 121 outputs the received sensor data to the walking state discrimination unit 122. The sensor data reception unit 121 allows the log storage unit 131 to store the received sensor data. For example, the sensor data reception unit 121 transmits a signal indicating that accumulation of log data is started to the log reading unit 132 via a signal line (not illustrated).

The walking state discrimination unit 122 is connected to the sensor data reception unit 121, the threshold storage unit 151, and the transmission unit 129. The walking state discrimination unit 122 acquires the sensor data from the sensor data reception unit 121. When acquiring the sensor data, the walking state discrimination unit 122 discriminates the walking state using any one of the two thresholds (the first threshold and the second threshold) stored in the threshold storage unit 151. The walking state discrimination unit 122 switches the operation mode according to a discrimination result of the walking state.

In the power saving mode, when the acceleration in the gravity direction (Z direction) exceeds the first threshold, the walking state discrimination unit 122 switches from the power saving mode to the discrimination mode. After switching to the discrimination mode, the walking state discrimination unit 122 discriminates the walking state using the acceleration in the travel direction (Y direction) included in the sensor data.

In a case where the acceleration in the travel direction (Y direction) exceeds the second threshold under a certain condition, the walking state discrimination unit 122 determines that the walk is started. For example, in a case where the acceleration in the travel direction (Y direction) exceeds the second threshold in a predetermined period, the walking state discrimination unit 122 determines that the walk is started. For example, in a case where the acceleration in the travel direction (Y direction) exceeds the second threshold a prescribed number of times or more in a predetermined period, the walking state discrimination unit 122 determines that a stable walk is started. For example, an elapsed time (about five seconds) of about several steps in a general walk is set as a discrimination time. If the elapsed time is set to five seconds and if it is determined to be the stable walk when steps of about three to five steps are measured, the prescribed number of times may be set to about three times. The discrimination time and the prescribed number of times may be set to initial setting values based on actual measurement values, or may be individually set for each user.

When determining that the walk is started, the walking state discrimination unit 122 switches from the discrimination mode to the walk measurement mode and transmits the sensor data including the acceleration data and the angular velocity data to the walk measurement device 13 via the transmission unit 129. When determining that the stable walk is started, the walking state discrimination unit 122 switches from the discrimination mode to the walk measurement mode and transmits the sensor data including the acceleration data and the angular velocity data to the walk measurement device 13 via the transmission unit 129.

For example, the walking state discrimination unit 122 performs mode switching in a sequence from the power saving mode to the discrimination mode, from the discrimination mode to the power saving mode, from the power saving mode to the discrimination mode, . . . , from the discrimination mode to the power saving mode and the like. For example, the walking state discrimination unit 122 may switch to the power saving mode in a case where the acceleration in the travel direction (Y direction) falls below the second threshold, or may switch to the power saving mode when a predetermined time elapses.

The change trend detection unit 123 detects the change trend of the peak values using the log data of the peak values of the acceleration in the travel direction (Y direction) in the discrimination mode. In the example in FIG. 5, the change trend detection unit 123 includes the log storage unit 131, the log reading unit 132, and the change trend determination unit 133.

The log storage unit 131 is connected to the sensor data reception unit 121 and the log reading unit 132. The log storage unit 131 stores the log data of the sensor data output by the sensor data reception unit 121. The log storage unit 131 is accessed by the log reading unit 132, and the log data of the sensor data stored therein is read.

The log reading unit 132 is connected to the log storage unit 131 and the change trend determination unit 133. The log reading unit 132 refers to the log storage unit 131 and reads the log data of the sensor data. For example, the log reading unit 132 accesses the log storage unit 131 at a predetermined timing, and reads the log data when a predetermined amount of the log data stored in the log storage unit 131 is accumulated. For example, the log reading unit 132 receives a signal indicating that the accumulation of the log data is started from the sensor data reception unit 121 via a signal line (not illustrated), and reads the log data from the log storage unit 131 when a predetermined time elapses after the accumulation of the log data is started. Note that, the timing at which the log reading unit 132 reads the log data from the log storage unit 131 is not limited to that in the above-described method, and may be optionally adjusted.

For example, the log reading unit 132 reads, from the log storage unit 131, the log data of the sensor data of a predetermined number of steps. The log reading unit 132 may read the log data at a timing when a preset time elapses after the log data of the sensor data is stored in the log storage unit 131. The log reading unit 132 outputs the read log data to the change trend determination unit 133.

The change trend determination unit 133 is connected to the log reading unit 132 and the threshold setting unit 152. The change trend determination unit 133 acquires the log data from the log reading unit 132. The change trend determination unit 133 determines a change trend of the walking waveform using the log data of the sensor data. The change trend determination unit 133 outputs a determination result of the change trend of the walking waveform to the threshold setting unit 152.

For example, the change trend determination unit 133 determines the change trend of the extreme values (also referred to as the peak values) of the waveform of the time-series data of the acceleration in the travel direction (Y direction) (walking waveform). For example, the change trend determination unit 133 detects the change trend such as upward, downward, and constant from the change in the peak values of the walking waveform. For example, the change trend determination unit 133 may be configured to detect a characteristic pattern caused when ascending/descending a slope, a staircase and the like from a change in the peak values.

For example, the change trend determination unit 133 determines the change trend (upward or downward) of the peak values using the log of the peak values of 10 steps stored in the log storage unit 131. When the peak values show an upward trend, the change trend determination unit 133 increases an absolute value of the threshold. In contrast, in a case where the peak values show a downward trend, the change trend determination unit 133 decreases the absolute value of the threshold.

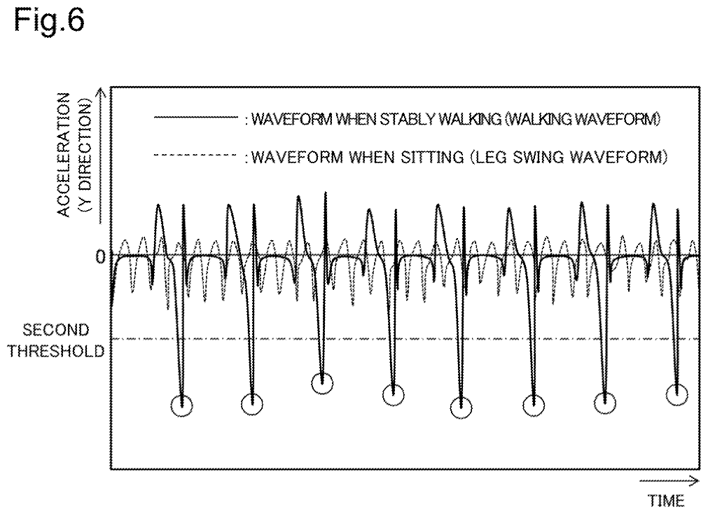

FIG. 6 is an example of the waveform of the time-series data of the acceleration in the travel direction (Y direction) (walking waveform). In FIG. 6, a waveform when stably walking (walking waveform) is indicated by a solid line. In FIG. 6, as a comparison, a waveform when swinging a leg while sitting (also referred to as a leg swing waveform) is indicated by a broken line. As in FIG. 6, the absolute value of the second threshold is set to a value larger than an absolute value of a smallest value out of minimum values of the leg swing waveform and smaller than an absolute value of a largest value out of the minimum values of the walking waveform. In the example in FIG. 6, the change trend determination unit 133 determines the change trend of the peak values caused by the contact of the heel (hereinafter referred to as heel contact) in the initial contact (1) in FIG. 4 described above. In FIG. 6, the peak value caused by the heel contact is the minimum value in a range enclosed by a circle.

For example, the change trend determination unit 133 performs regression analysis on the peak values of a predetermined number of steps using a predetermined regression model. As an example, the change trend determination unit 133 performs linear regression on the peak values of a predetermined number of steps using a linear function to determine the change trend of the peak values. As an example, the change trend determination unit 133 performs regression analysis on the peak values of a predetermined number of steps using a function such as a quadratic function and a cubic function to determine the change trend of the peak values. As an example, the change trend determination unit 133 may determine the change trend of the peak values by performing regression analysis on the peak values of a predetermined number of steps using a function such as a fractional function, an irrational function, a logarithmic function, an exponential function, a trigonometric function, an inverse trigonometric function, and a hyperbolic function.

The threshold change unit 125 changes the second threshold based on a detection result by the change trend detection unit 123. In the example in FIG. 5, the threshold change unit 125 includes the threshold storage unit 151 and the threshold setting unit 152.

The threshold storage unit 151 stores the two thresholds (the first threshold and the second threshold) used for discriminating the walking state by the walking state discrimination unit 122. The first threshold out of the two thresholds is the threshold set for the acceleration in the gravity direction (Z direction) used when the power saving mode is switched to the discrimination mode. The second threshold out of the two thresholds is the threshold set for the acceleration in the gravity direction (Y direction) used after the power saving mode is switched to the discrimination mode. The second threshold is reset by the threshold setting unit 152.

The threshold setting unit 152 is connected to the change trend determination unit 133 and the threshold storage unit 151. The threshold setting unit 152 resets the second threshold regarding the acceleration in the travel direction (Y direction) according to the determination result by the change trend determination unit 133.

In general, the acceleration in the travel direction of the walker changes according to a condition of a road surface and a state of the walker. The condition of the road surface varies among concrete, carpet, lawn and the like. The state of the walker changes according to a fatigue level and the like. A condition of the shoes such as an abrasion condition of sole and heel also affects the acceleration in the travel direction of the walker. In a case of determining the walking state based on the acceleration in the travel direction, if the threshold used for the determination is fixed, there is a case of no activation or erroneous activation. Therefore, the threshold setting unit 152 resets the second threshold based on the change trend of the peak values of the time-series data of the acceleration in the travel direction (Y direction) (walking waveform) of the walker changing according to the condition of the road surface and the state of the walker.

The transmission unit 129 is connected to the walking state discrimination unit 122 and the walk measurement device 13. The transmission unit 129 acquires the discrimination result and the sensor data from the walking state discrimination unit 122. The transmission unit 129 transmits the acquired discrimination result and sensor data to the walk measurement device 13. For example, when the discrimination result indicating the stable walk is transmitted from the transmission unit 129 to the walk measurement device 13, the walk measurement by the walk measurement device 13 is started. When the discrimination result indicating that the walk is stopped is transmitted from the transmission unit 129 to the walk measurement device 13, the walk measurement by the walk measurement device 13 is stopped. It is possible to configure such that the discrimination result indicating the stable walk is transmitted from the transmission unit 129 to the walk measurement device 13, and the walk measurement device 13 directly receives the sensor data from the data acquisition device according to the discrimination result. It is also possible to configure such that the walk measurement device 13 starts the walk measurement using the sensor data as the trigger without transmitting the discrimination result.

The configuration of the determination device 12 is described above. The configuration of the determination device 12 in FIG. 5 is an example, and the configuration of the determination device 12 of the present example embodiment is not limited to this form.

[Data Acquisition Device]

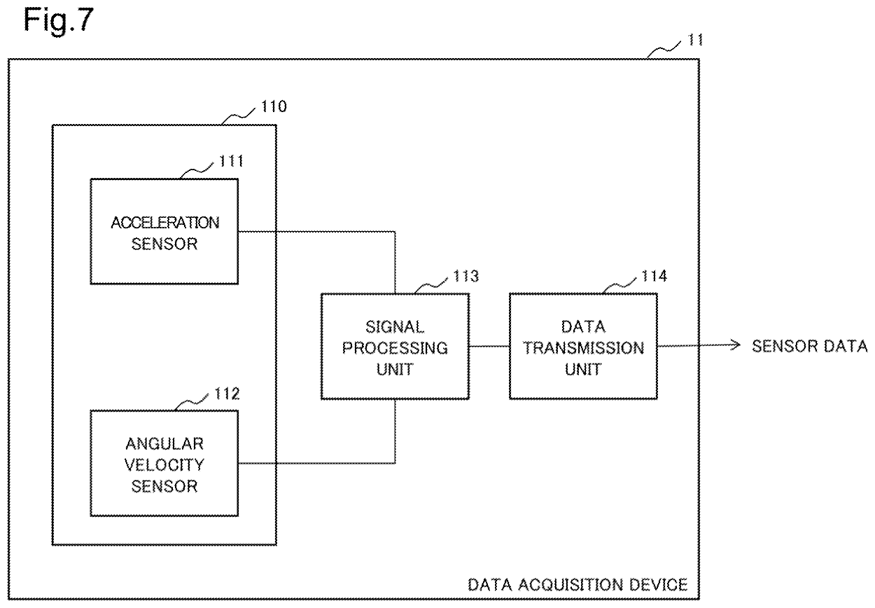

Next, the data acquisition device 11 provided in the walk measurement system 1 is described with reference to the drawings. FIG. 7 is a block diagram illustrating an example of a configuration of the data acquisition device 11. The data acquisition device 11 includes an acceleration sensor 111, an angular velocity sensor 112, a signal processing unit 113, and a data transmission unit 114. The acceleration sensor 111 and the angular velocity sensor 112 form a sensor 110. For example, the data acquisition device 11 is achieved by an IMU.

The acceleration sensor 111 is the sensor that measures acceleration in three-axis directions. As for the acceleration in the three-axis directions measured by the acceleration sensor 111, for example, the acceleration sensor 111 measures the acceleration in the three-axis directions set in the lateral direction (X direction), the travel direction (Y direction), and the gravity direction (Z direction). The acceleration sensor 111 outputs the measured acceleration to the signal processing unit 113.

The angular velocity sensor 112 is the sensor that measures the angular velocity. The angular velocity sensor 112 outputs the measured angular velocity to the signal processing unit 113.

The signal processing unit 113 acquires the acceleration and the angular velocity from the acceleration sensor 111 and the angular velocity sensor 112, respectively. The signal processing unit 113 converts the acquired acceleration and angular velocity into digital data, and outputs the converted digital data (sensor data) to the data transmission unit 114. The sensor data at least includes the acceleration data obtained by converting the acceleration of analog data into digital data and the angular velocity data obtained by converting the angular velocity of analog data into digital data. The sensor data may also include an acquisition time of raw data of the acceleration and angular velocity. The signal processing unit may be configured to output sensor data obtained by correcting a mounting error, temperature, and linearity of the acquired raw data of the acceleration and angular velocity.

The data transmission unit 114 acquires the sensor data from the signal processing unit 113. The data transmission unit 114 transmits the acquired sensor data to the determination device 12. The data transmission unit 114 may transmit the sensor data to the determination device 12 by wire such as a cable or a conductive wire, or may transmit the sensor data to the determination device 12 by wireless communication. For example, the data transmission unit 114 may be configured to transmit the sensor data to the determination device 12 via a wireless communication function (not illustrated) conforming to standards such as Bluetooth (registered trademark) or Wi-Fi (registered trademark). In a case where the sensor data is directly transmitted from the data transmission unit 114 to the walk measurement device 13, it may be configured that the sensor data is transmitted from the data transmission unit 114 to the walk measurement device 13 by wired communication or wireless communication.

An example of the configuration of the data acquisition device 11 is described above. The configuration in FIG. 7 is an example, and the configuration of the data acquisition device 11 provided in the walk measurement system 1 of the present example embodiment is not limited to this form as-is.

(Operation)

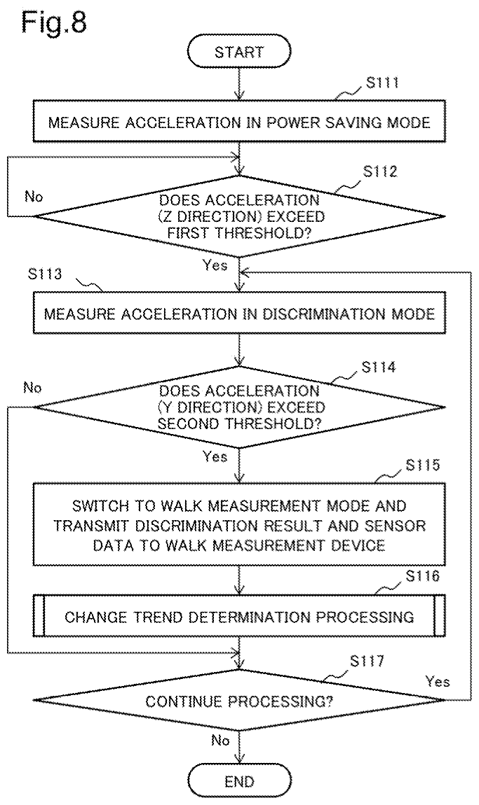

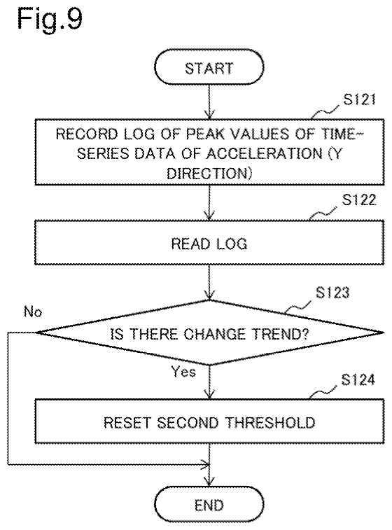

Next, the operation of the walk measurement system 1 of the present example embodiment is described with reference to the drawings. Hereinafter, operation mode switching processing (mode switching processing: FIG. 8) and determination processing of a walking trend (walking trend determination processing: FIG. 9) are described.

[Mode Switching]

FIG. 8 is a flowchart for illustrating the mode switching processing of switching the operation mode from the power saving mode to the discrimination mode and further to the walk measurement mode according to an operation of the user. In the description along the flowchart in FIG. 8, the determination device 12 is described as a subject of the operation.

In FIG. 8, the determination device 12 first measures the acceleration in the power saving mode (step S111).

Next, the determination device 12 switches the operation mode according to a relationship between the acceleration in the gravity direction (Z direction) and the first threshold (step S112). For example, a process at step S112 is performed under a certain condition such as a timing when it reaches a predetermined time or a timing when a predetermined time elapses.

When the acceleration in the gravity direction (Z direction) exceeds the first threshold (Yes at step S112), the determination device 12 switches from the power saving mode to the discrimination mode and measures the acceleration (step S113). When the power saving mode is switched to the discrimination mode, a measurement speed of the acceleration by the data acquisition device 11 is switched from a low speed to a high speed. It is also possible to configure such that measurement frequency of the acceleration by the data acquisition device 11 is switched from low frequency to high frequency when the power saving mode is switched to the discrimination mode. In contrast, in a case where the acceleration in the gravity direction (Z direction) does not exceed the first threshold (Yes at step S112), step S112 is repeated.

After step S113, the determination device 12 switches the operation mode according to a relationship between the acceleration in the travel direction (Y direction) and the second threshold (step S114). For example, a process at step S114 is performed under a certain condition such as a timing when it reaches a predetermined time or a timing when a predetermined time elapses.

In a case where the acceleration in the travel direction (Y direction) exceeds the second threshold (Yes at step S114) when the acceleration is measured in the discrimination mode, the determination device 12 switches from the discrimination mode to the walk measurement mode (step S115). When switching to the walk measurement mode, the determination device 12 transmits the discrimination result indicating the stable walk and the sensor data to the walk measurement device. After switching to the walk measurement mode, the determination device 12 continuously transmits the sensor data to the walk measurement device. In contrast, in a case where the acceleration in the travel direction (Y direction) does not exceed the second threshold (No at step S114) when the acceleration is measured in the discrimination mode, the procedure shifts to step S117.

After step S115, the determination device 12 executes change trend determination processing (FIG. 9) (step S116). In the change trend determination processing, the determination device 12 detects the change trend of the peak values from the walking waveform, and resets the second threshold based on the detected change trend.

In a case where the processing is continued (Yes at step S117), the procedure returns to step S113. In contrast, in a case where the processing is not continued (No at step S117), the processing along the flowchart in FIG. 8 ends.

The mode switching for switching the operation mode according to the operation of the user is described above. The flowchart in FIG. 8 is an example, and the switching of the operation mode by the determination device 12 of the present example embodiment is not limited to this procedure.

[Change Trend Determination Processing]

FIG. 9 is a flowchart for illustrating the change trend determination processing executed when the operation mode is switched to the walk measurement mode. In the description along the flowchart in FIG. 9, the determination device 12 is described as a subject of the operation.

In FIG. 9, the determination device 12 first stores the log of the extreme values (peak values) of the time-series data of the acceleration in the travel direction (Y direction) (walking waveform) (step S121). For example, the determination device 12 at least stores the log used when determining the change trend of the peak values (for example, 10 steps).

Next, the determination device 12 reads the log used for determining the change trend of the peak values (step S122).

Here, the determination device 12 determines the change trend of the peak values (step S123). In a case where the change trend is detected (Yes at step S123), the determination device 12 resets the second threshold based on the change trend of the peak values (step S124). In contrast, in a case where the change trend of the peak values is not detected (No at step S123), the processing along the flowchart in FIG. 9 ends without resetting the second threshold.

The change trend determination processing by the determination device 12 of the present example embodiment is described above. The processing along the flowchart in FIG. 9 is an example, and the change trend determination processing by the determination device 12 is not limited to this procedure.

As described above, the determination device of the present example embodiment is provided with the sensor data reception unit, the walking state discrimination unit, the change trend detection unit, the threshold change unit, and the transmission unit. The sensor data reception unit receives the sensor data including the acceleration in the travel direction of the user and the gravity direction. The walking state discrimination unit switches to the discrimination mode when the acceleration in the gravity direction exceeds the first threshold in the power saving mode, and switches to the walk measurement mode when the acceleration in the travel direction exceeds the second threshold in the discrimination mode. The change trend detection unit detects the change trend of the peak values using the log data of the peak values of the acceleration in the travel direction in the discrimination mode. The threshold change unit changes the second threshold based on the detection result by the change trend detection unit. The transmission unit transmits the sensor data in the walk measurement mode.

In one aspect of the present example embodiment, the change trend determination unit determines the change trend of the peak values using the log data of the peak values associated to the heel contact of the user.

In one aspect of the present example embodiment, the change trend detection unit includes the log storage unit, the log reading unit, and the change trend determination unit. The log storage unit stores the log data of the peak values of the acceleration in the travel direction in the discrimination mode. The log reading unit reads the log data of the peak values stored in the log storage unit. The change trend determination unit determines the change trend of the peak values using the log data of the peak values.

In one aspect of the present example embodiment, the threshold change unit includes the threshold storage unit and the threshold setting unit. The threshold storage unit stores the first threshold set for the acceleration in the gravity direction and the second threshold set for the acceleration in the travel direction. The threshold setting unit sets the second threshold stored in the threshold storage unit based on the determination result by the change trend detection unit.

The walk measurement system of the present example embodiment is provided with the data acquisition device, the determination device, and the walk measurement device. The data acquisition device is arranged in the footwear of the user, generates the sensor data according to the operation mode switched by the walking state discrimination unit, and transmits the generated sensor data to the determination device. The walk measurement device receives the sensor data transmitted from the data acquisition device, and measures the walk of the user using the received sensor data.

The determination device of the present example embodiment may flexibly change the second threshold set for the acceleration in the travel direction based on the determination result of the change trend of the peak values of the acceleration in the travel direction. Therefore, according to the determination device of the present example embodiment, it is possible to achieve high efficiency and low power consumption of the walk measurement while flexibly responding to the change in the walking state.

### Second Example Embodiment

Next, a walk measurement system according to a second example embodiment of the present invention is described with reference to the drawings. The walk measurement system of the present example embodiment determines a change trend of peak values by performing linear regression on extreme values (also referred to as peak values) of a waveform of time-series data of acceleration in a travel direction (Y direction) (walking waveform) and changes a second threshold.

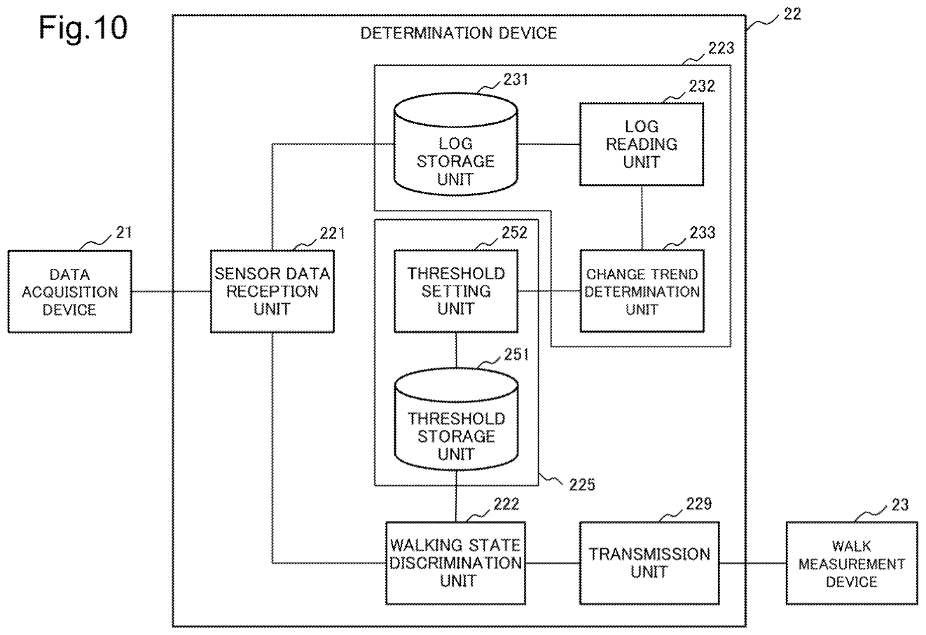

FIG. 10 is a block diagram illustrating an example of a configuration of the walk measurement system of the present example embodiment. The walk measurement system of the present example embodiment is provided with a data acquisition device 21, a determination device 22, and a walk measurement device 23. The determination device 22 includes a sensor data reception unit 221, a walking state discrimination unit 222, a change trend detection unit 223, a threshold change unit 225, and a transmission unit 229. As an example, the change trend detection unit 223 in FIG. 10 includes a log storage unit 231, a log reading unit 232, and a change trend determination unit 233. As an example, the threshold change unit 225 in FIG. 10 includes a threshold storage unit 251 and a threshold setting unit 252. The walk measurement system of the present example embodiment is similar to the walk measurement system 1 of the first example embodiment except for an operation of the change trend determination unit 233. Therefore, in the following, difference from the walk measurement system 1 of the first example embodiment is mainly described.

The change trend determination unit 233 determines the change trend of the peak values using a slope of a linear regression line obtained by performing linear regression on the extreme values (also referred to as the peak values) of the waveform of the time-series data of the acceleration in the travel direction (Y direction) (walking waveform). For example, the change trend determination unit 233 determines the change trend (upward or downward) of the peak values using a log of the peak values of 10 steps stored in the log storage unit 231.

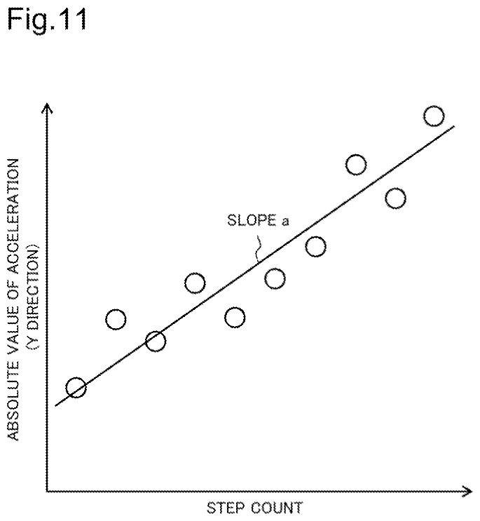

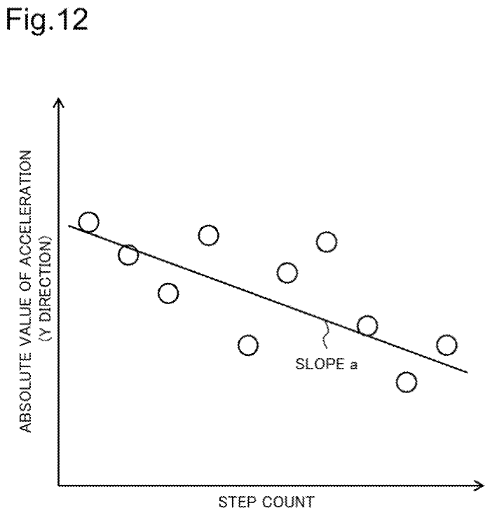

FIGS. 11 and 12 are graphs illustrating an example of a relationship between a step count used when the change trend determination unit 233 discriminates a walking state and absolute values of the extreme values (peak values) of the time-series data of the acceleration in the travel direction (Y direction) (step number waveform). FIGS. 11 and 12 are graphs in which the peak values are plotted with respect to the step count of 10 steps. FIG. 11 is an example in which the change trend of the peak values is upward. FIG. 12 is an example in which the change trend of the peak values is downward. In FIGS. 11 and 12, a linear regression line of the step count and the peak values is indicated by a solid line. Note that, the regression lines illustrated in FIGS. 11 and 12 are not obtained by accurate linear regression on the plots of the peak values with respect to the step count.

In a case where a slope a of the linear regression line is positive, the change trend determination unit 233 notifies the threshold setting unit 252 that the change trend is upward. In contrast, in a case where the change trend of the peak values is downward, the change trend determination unit 233 notifies the threshold setting unit 252 that the change trend is downward. In either case, the change trend determination unit 233 outputs the slope a of the linear regression line to the threshold setting unit 252.

For example, the change trend determination unit 233 determines the change trend of the peak values by the slope a (acceleration/the number of steps) of the straight line obtained by performing linear regression on the peak values of a predetermined number of steps. In a case where the slope a is positive, the change trend determination unit 233 determines that the change trend is upward and transmits the slope a to the threshold setting unit 252. In contrast, in a case where the slope a is negative, the change trend determination unit 233 determines that the change trend is downward and transmits the slope a to the threshold setting unit 252. In a case where the slope a is 0, it is possible that the change trend determination unit 233 transmits a fact that there is no change trend to the threshold setting unit 252, or does not notify the same of anything. The change trend determination unit 233 may perform regression on the peak values of a predetermined number of steps using a function such as a polynomial function such as a quadratic function or a cubic function, a fractional function, an irrational function, a logarithmic function, an exponential function, a trigonometric function, an inverse trigonometric function, or a hyperbolic function.

The threshold setting unit 252 receives the notification indicating whether the change trend of the peak values is upward or downward. When receiving the notification that the change trend is upward, the threshold setting unit 252 increases an absolute value of the second threshold. In contrast, when receiving the notification that the change trend is downward, the threshold setting unit 252 decreases the absolute value of the second threshold.

For example, in a case where the slope a (acceleration/the number of steps) of the straight line obtained by performing linear regression on the peak values of a predetermined number of steps is used, the threshold setting unit 252 receives the slope a of the regression line from the change trend determination unit 233, and resets the second threshold using following equation 1. Note that, in equation 1, the threshold before the change is denoted by Bo, and the threshold after the change is denoted by Bi. For example, the threshold setting unit 352 may adjust the increase or decrease of the second threshold by weighting the slope a.

$${B}_{1} = {B}_{0} + a \qquad ( 1 )$$

The configuration of the walk measurement system of the present example embodiment is described above. The configuration in FIG. 10 is an example, and the configuration of the walk measurement system of the present example embodiment is not limited to this form.

(Operation)

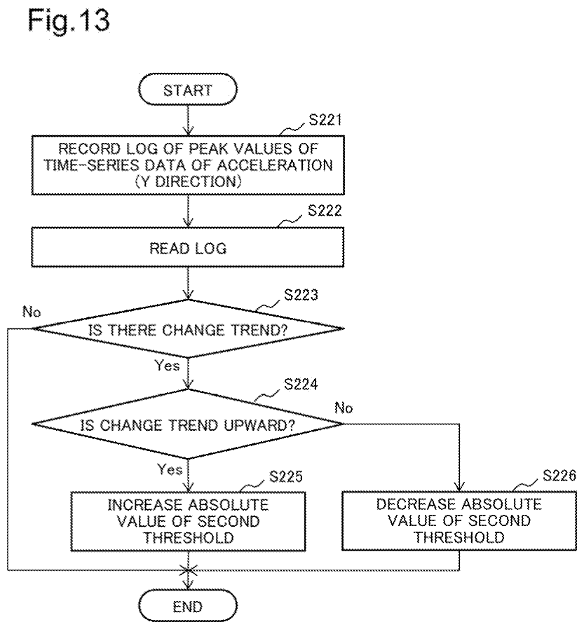

Next, an operation of the walk measurement system of the present example embodiment is described with reference to the drawings. Hereinafter, operation mode switching processing similar to that of the first example embodiment is omitted and determination processing of a walking trend (walking trend determination processing: FIG. 13) is described.

[Change Trend Determination Processing]

FIG. 13 is a flowchart for illustrating a change trend determination processing executed when an operation mode is switched to a walk measurement mode. In the description along the flowchart in FIG. 13, the determination device 22 is described as a subject of the operation.

In FIG. 13, the determination device 22 first stores the log of the extreme values (peak values) of the time-series data of the acceleration in the travel direction (Y direction) (walking waveform) (step S221). For example, the determination device 22 at least stores the log used for determining the change trend of the peak values (for example, 10 steps).

Next, the determination device 22 reads the log used for determining the change trend of the peak values (step S222).

Here, the determination device 22 determines whether there is the change trend of the peak values (step S223). In a case where the change trend of the peak values is detected (Yes at step S223), the determination device 22 determines whether the change trend is upward (step S224). In contrast, in a case where the change trend is not detected (No at step S223), the processing along the flowchart in FIG. 13 ends.

In a case where the change trend of the peak values is upward (Yes at step S224), the determination device 22 increases the absolute value of the second threshold (step S225). In contrast, in a case where the change trend of the peak values is downward (No at step S224), the determination device 22 decreases the absolute value of the second threshold (step S226). At steps S225 and S226, the processing along the flowchart in FIG. 13 ends.

The change trend determination processing by the determination device 22 of the present example embodiment is described above. The processing along the flowchart in FIG. 13 is an example, and the change trend determination processing by the determination device 22 is not limited to this procedure.

As described above, the change trend detection unit determines that the change trend of the peak values is upward in a case where the slope of the regression line obtained by performing linear regression on the log data of the peak values is positive, and determines that the change trend of the peak values is downward in a case where the slope of the regression line is negative. The threshold change unit increases the absolute value of the second threshold in a case where it is determined that the change trend of the peak values is upward, and decreases the absolute value of the second threshold in a case where it is determined that the change trend of the peak values is downward.

The determination device of the present example embodiment determines the change trend of the peak values according to whether the slope of the regression line is positive or negative. Therefore, according to the determination device of the present example embodiment, the change trend of the peak values may be determined with a clear determination criterion.

### Third Example Embodiment

Next, a walk measurement system according to a second example embodiment of the present invention is described with reference to the drawings. The walk measurement system of the present example embodiment determines a change trend of peak values by performing linear regression on extreme values (also referred to as peak values) of a waveform of time-series data of acceleration in a travel direction (Y direction) (walking waveform) and changes a second threshold. The walk measurement system of the present example embodiment is different from that of the second example embodiment in determining that, when the change trend of the peak values is within a constant determination range, the change trend is constant and not changing a threshold.

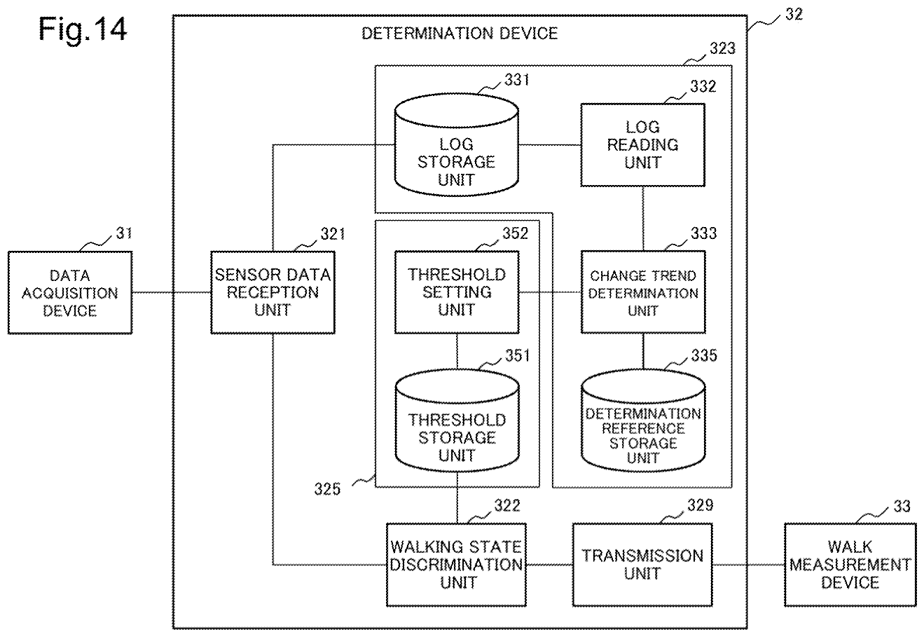

FIG. 14 is a block diagram illustrating an example of a configuration of the walk measurement system of the present example embodiment. The walk measurement system of the present example embodiment is provided with a data acquisition device 31, a determination device 32, and a walk measurement device 33. The determination device 32 includes a sensor data reception unit 321, a walking state discrimination unit 322, a change trend detection unit 323, a threshold change unit 325, and a transmission unit 329. As an example, the change trend detection unit 323 in FIG. 14 includes a log storage unit 331, a log reading unit 332, a change trend determination unit 333, and a determination criterion storage unit 335. As an example, the threshold change unit 325 in FIG. 14 includes a threshold storage unit 351 and a threshold setting unit 352. The walk measurement system of the present example embodiment is similar to the walk measurement system of the second example embodiment except that this includes the determination criterion storage unit 335, and the change trend determination unit 333 determines the change trend of the peak values based on a determination criterion stored in the determination criterion storage unit 335. Therefore, in the following, difference from the walk measurement system of the second example embodiment is mainly described.

The determination criterion storage unit 335 stores the constant determination range in which it is determined that a slope of a straight line (hereinafter, also referred to as a regression line) obtained when linear regression is performed on the peak values of the walking waveform is constant. For example, the determination criterion storage unit 335 stores a lower limit slope d and an upper limit slope u between which it is determined that the slope of the regression line is constant. Note that d and u are real numbers, and u is larger than d.

When receiving a log of the peak values from the log reading unit 332, the change trend determination unit 333 determines the change trend of the peak values with reference to the constant determination range stored in the determination criterion storage unit 335. In a case where the slope a of the regression line is within the constant determination range (d<a<u), the change trend determination unit 333 determines that the change trend of the peak values is constant. Then, the change trend determination unit 333 determines that the change trend of the peak values is upward in a case where the slope a of the regression line exceeds the upper limit slope u (u<a), and determines that the change trend of the peak values is downward in a case where the slope a of the regression line falls below the lower limit slope d (a<d). In a case where the slope a of the regression line matches a boundary value of the constant determination range (a=u or a=d), the change trend of the peak values may be determined to be constant or determined to change based on a determination criterion set in advance.

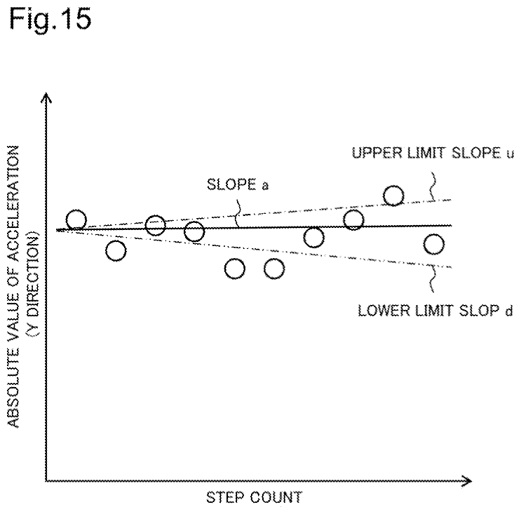

FIG. 15 is a graph illustrating an example of a relationship between a step count used when the change trend determination unit 333 discriminates a walking state and absolute values of the extreme values (peak values) of the time-series data of the acceleration in the travel direction (Y direction) (step number waveform). FIG. 15 is a graph in which the peak values are plotted with respect to the step count of 10 steps. In FIG. 15, a linear regression line of the step count and the peak values is indicated by a solid line. FIG. 15 also illustrates a straight line of the lower limit slope d (dashed two-dotted line) and a straight line of the upper limit slope u (dashed-dotted line) between which it is determined that the slope a of the linear regression line is constant. Note that the regression line illustrated in FIG. 15 is not obtained by accurate linear regression on the plots of the peak values with respect to the step count.

In the example in FIG. 15, the slope a of the linear regression line is within the constant determination range (d<a<u), so that the change trend determination unit 333 determines that the change trend of the peak values is constant. In a case where the change trend of the peak values is constant, it is possible that the change trend determination unit 333 transmits a notification that the peak values are constant or does not transmit this. At that time, the second threshold is not reset.

In a case where the slope a of the linear regression line exceeds the upper limit slope u (u<a), the change trend determination unit 333 notifies the threshold setting unit 352 that the change trend is upward. In contrast, in a case where the slope a of the linear regression line falls below the lower limit slope d (a<d), the change trend determination unit 333 notifies the threshold setting unit 352 that the change trend is downward. In either case, the change trend determination unit 333 may be configured to output the slope a of the linear regression line to the threshold setting unit 352 and change the second threshold according to a value of the slope a.

The threshold setting unit 352 receives the notification indicating whether the change trend of the peak values is upward or downward from the change trend determination unit 333. For example, the threshold setting unit 352 receives the slope a (acceleration/the number of steps) of the regression line from the change trend determination unit 333 as the notification of whether the change trend is upward or downward. When receiving the notification that the change trend is upward, the threshold setting unit 352 increases an absolute value of the second threshold according to the received notification. In contrast, when receiving the notification that the change trend is downward, the threshold setting unit 352 decreases the absolute value of the second threshold according to the received notification. The threshold setting unit 352 allows the threshold storage unit 351 to store the reset second threshold.

For example, in a case where the slope a (acceleration/the number of steps) of the straight line obtained by performing linear regression on the peak values of a predetermined number of steps is used, the threshold setting unit 352 receives the slope a of the regression line from the change trend determination unit 333, and resets the second threshold using equation 1 described above. For example, the threshold setting unit 352 may adjust the increase or decrease of the second threshold by weighting the slope a.

The configuration of the walk measurement system of the present example embodiment is described above. The configuration in FIG. 14 is an example, and the configuration of the walk measurement system of the present example embodiment is not limited to this form.

(Operation)

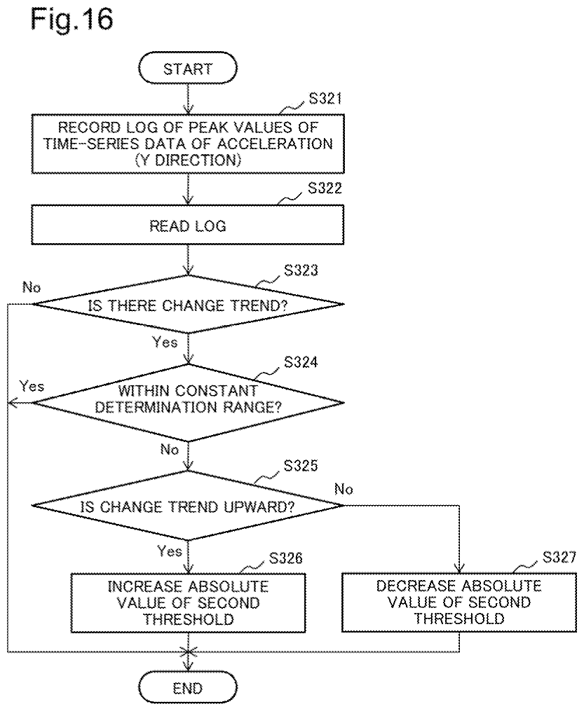

Next, an operation of the walk measurement system of the present example embodiment is described with reference to the drawings. Hereinafter, operation mode switching processing similar to that of the first example embodiment is omitted and determination processing of a walking trend (walking trend determination processing: FIG. 16) is described.

[Change Trend Determination Processing]

FIG. 16 is a flowchart for illustrating a change trend determination processing executed when an operation mode is switched to a walk measurement mode. In the description along the flowchart in FIG. 16, the determination device 32 is described as a subject of the operation.

In FIG. 16, the determination device 32 first stores the log of the extreme values (peak values) of the time-series data of the acceleration in the travel direction (Y direction) (walking waveform) (step S321). For example, the determination device 32 at least stores the log used when determining the change trend of the peak values (for example, 10 steps).

Next, the determination device 32 reads the log used for determining the change trend of the peak values (step S322).

Here, the determination device 32 determines whether there is the change trend of the peak values (step S323). In a case where the change trend is detected (Yes at step S323), the determination device 32 performs linear regression on the step count and the peak values, and determines whether the slope of the regression line is within the constant determination range (step S324). In contrast, in a case where the change trend is not detected (No at step S323), the processing along the flowchart in FIG. 16 ends.

In a case where the slope of the regression line is within the constant determination range (Yes at step S324), the processing along the flowchart in FIG. 16 ends. In contrast, in a case where the slope of the regression line is without the constant determination range (No at step S324), the determination device 32 determines whether the change trend of the peak values is upward (step S325).

In a case where the change trend of the peak values is upward (Yes at step S325), the determination device 32 increases the absolute value of the second threshold (step S326). In contrast, in a case where the change trend of the peak values is downward (No at step S325), the determination device 32 decreases the absolute value of the second threshold (step S327). At steps S326 and S327, the processing along the flowchart in FIG. 16 ends.

The change trend determination processing by the determination device 32 of the present example embodiment is described above. The processing along the flowchart in FIG. 16 is an example, and the change trend determination processing by the determination device 32 is not limited to this procedure.

As described above, the determination device of the present example embodiment is provided with the determination criterion storage unit that stores the determination criterion regarding the range of the slope of the regression line in which the change trend of the peak values is determined to be constant. In a case where the slope of the regression line is within the range of the determination criterion, the change trend detection unit determines that the change trend of the peak values is constant. The threshold change unit does not change the second threshold in a case where it is determined that the change trend of the peak values is constant.

The determination device of the present example embodiment does not change the second threshold in a case where it is determined that the change trend of the peak values is constant. Therefore, according to the determination device of the present example embodiment, the change of the second threshold may be omitted in a case where the change trend of the peak values to such an extent as to switch the operation mode is not observed.

### Fourth Example Embodiment

Next, a walk measurement system according to a fourth example embodiment of the present invention is described with reference to the drawings. The walk measurement system of the present example embodiment determines a change trend of extreme values (also referred to as peak values) of a waveform of time-series data of acceleration in a travel direction (Y direction) (walking waveform) by using a learning model. The walk measurement system of the present example embodiment is different from that of the first example embodiment in that the change trend of the peak values is determined using the learning model.

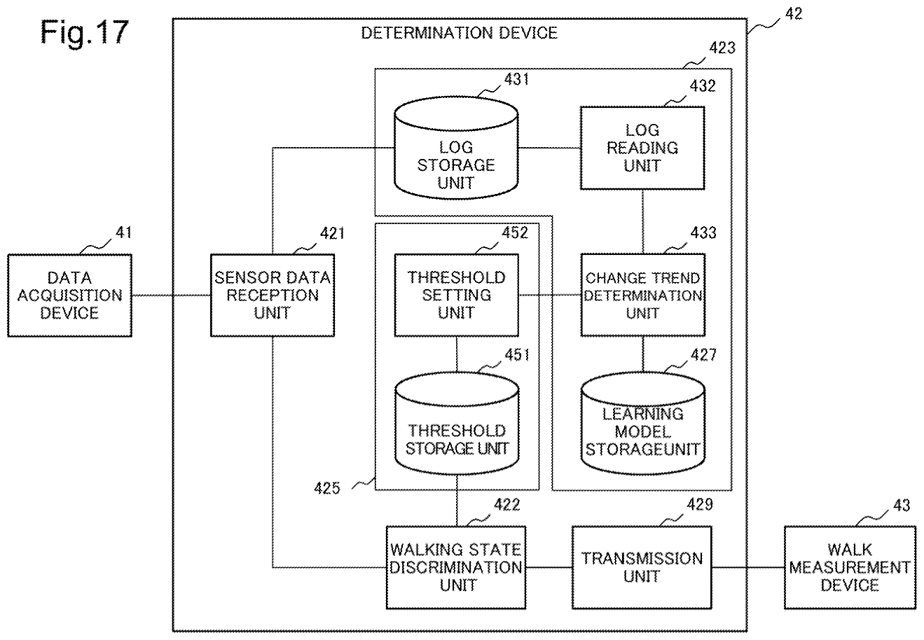

FIG. 17 is a block diagram illustrating an example of a configuration of the walk measurement system of the present example embodiment. The walk measurement system of the present example embodiment is provided with a data acquisition device 41, a determination device 42, and a walk measurement device 43. The determination device 42 includes a sensor data reception unit 421, a walking state discrimination unit 422, a change trend detection unit 423, a threshold change unit 425, and a transmission unit 429. As an example, the change trend detection unit 423 in FIG. 17 includes a log storage unit 431, a log reading unit 432, a change trend determination unit 433, and a learning model storage unit 427. As an example, the threshold change unit 425 in FIG. 17 includes a threshold storage unit 451 and a threshold setting unit 452. The walk measurement system of the present example embodiment is similar to the walk measurement system of the first example embodiment except that the walk measurement system of the present example embodiment includes the learning model storage unit 427, and the change trend determination unit 433 determines the change trend of the peak values based on the learning model stored in the learning model storage unit 427. Therefore, in the following, difference from the walk measurement system of the first example embodiment is mainly described.

The learning model storage unit 427 stores the learning model for determining the change trend of the peak values based on a pattern of the peak values of the walking waveform. The learning model stored in the learning model storage unit 427 may be a model given in advance or a model learned by machine learning. The learning model stored in the learning model storage unit 427 is not particularly limited.

When receiving a log of the peak values from the log reading unit 432, the change trend determination unit 433 determines the change trend of the peak values using the learning model stored in the learning model storage unit 427. The change trend determination unit 433 transmits the change trend of the peak values determined using the learning model to the threshold setting unit 452.

The threshold setting unit 452 receives a notification indicating whether the change trend of the peak values is upward or downward from the change trend determination unit 433. When receiving the notification that the change trend is upward, the threshold setting unit 452 increases an absolute value of a second threshold according to the received notification. In contrast, when receiving the notification that the change trend is downward, the threshold setting unit 452 decreases the absolute value of the second threshold according to the received notification. The threshold setting unit 452 allows the threshold storage unit 451 to store the reset second threshold.

The configuration of the walk measurement system of the present example embodiment is described above. The configuration in FIG. 17 is an example, and the configuration of the walk measurement system of the present example embodiment is not limited to this form.

(Operation)

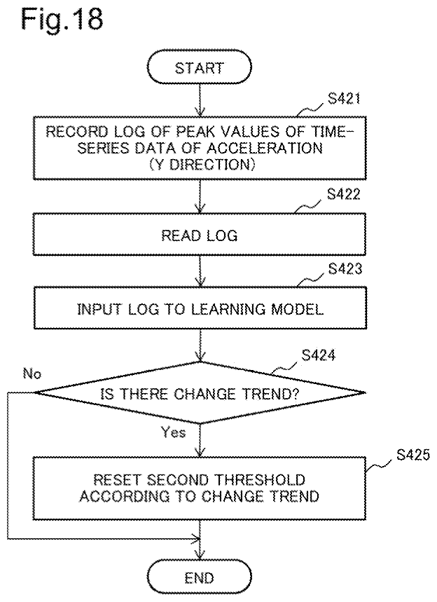

Next, an operation of the walk measurement system of the present example embodiment is described with reference to the drawings. Hereinafter, operation mode switching processing similar to that of the first example embodiment is omitted and determination processing of a walking trend (walking trend determination processing: FIG. 18) is described.

[Change Trend Determination Processing]

FIG. 18 is a flowchart for illustrating change trend determination processing executed when an operation mode is switched to a walk measurement mode. In the description along the flowchart in FIG. 18, the determination device 42 is described as a subject of the operation.

In FIG. 18, the determination device 42 first stores the log of the extreme values (peak values) of the time-series data of the acceleration in the travel direction (Y direction) (walking waveform) (step S421). For example, the determination device 42 at least stores the log used for determining the change trend of the peak values (for example, 10 steps).

Next, the determination device 42 reads the log used for determining the change trend of the peak values (step S422).

Next, the determination device 42 inputs the read log to the learning model (step S423).

Here, the determination device 42 determines whether there is the change trend of the peak values (step S424). In a case where the change trend of the peak values is detected (Yes at step S424), the determination device 42 resets the second threshold according to the detected change trend (step S425), and the processing along the flowchart in FIG. 18 ends. In contrast, in a case where the change trend is not detected (No at step S424), the processing along the flowchart in FIG. 18 ends.

The change trend determination processing by the determination device 42 of the present example embodiment is described above. The processing along the flowchart in FIG. 18 is an example, and the change trend determination processing by the determination device 42 is not limited to this procedure.

As described above, the determination device of the present example embodiment is provided with the learning model storage unit that stores the learning model regarding the change trend of the peak values. The change trend detection unit detects the change trend of the peak values by applying the log data of the peak values to the learning model.

The determination device of the present example embodiment uses the learning model to determine the change trend according to not only the increase or decrease of the peak values but also a characteristic pattern observed in the change of the peak values. Therefore, according to the present example embodiment, as compared with the first example embodiment, it is possible to achieve high efficiency and low power consumption of the walk measurement while more flexibly responding to a change in walking state.

### Fifth Example Embodiment

Next, a walk measurement system according to a fifth example embodiment of the present invention is described with reference to the drawings. The walk measurement system of the present example embodiment is different from that of the first example embodiment in notifying a user of a determination result of a change trend of extreme values (also referred to as peak values) of a waveform of time-series data of acceleration in a travel direction (Y direction) (walking waveform) using a learning model.

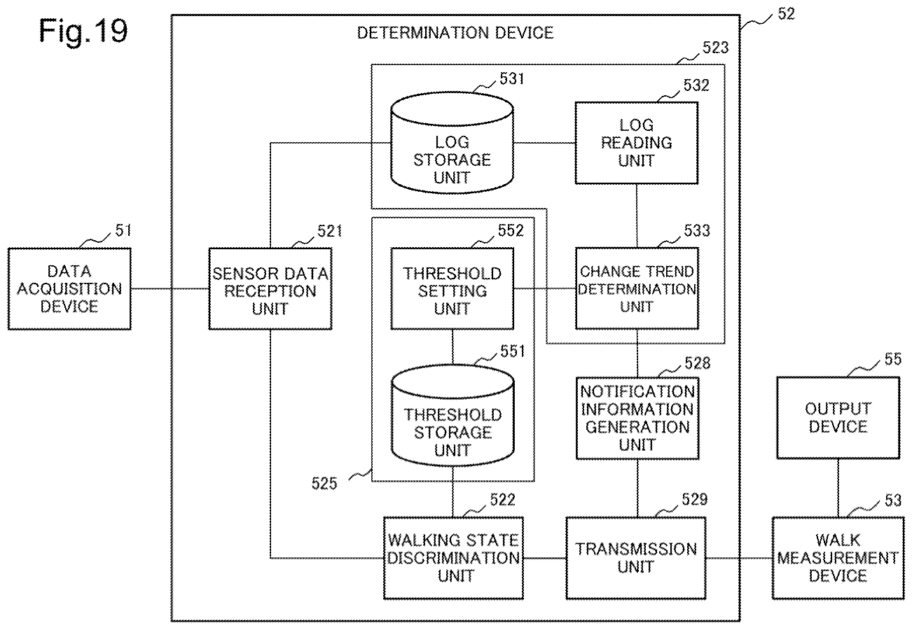

FIG. 19 is a block diagram illustrating an example of a configuration of the walk measurement system of the present example embodiment. The walk measurement system of the present example embodiment is provided with a data acquisition device 51, a determination device 52, a walk measurement device 53, and an output device 55. The determination device 52 includes a sensor data reception unit 521, a walking state discrimination unit 522, a change trend detection unit 523, a threshold change unit 525, a notification information generation unit 528, and a transmission unit 529. As an example, the change trend detection unit 523 in FIG. 19 includes a log storage unit 531, a log reading unit 532, and a change trend determination unit 533. As an example, the threshold change unit 525 in FIG. 17 includes a threshold storage unit 551 and a threshold setting unit 552. The walk measurement system of the present example embodiment is similar to the walk measurement system of the first example embodiment except that the notification information generation unit 528 generates notification information regarding a determination result by the change trend determination unit 533, and the notification information is output from the output device 55. Therefore, in the following, difference from the walk measurement system of the first example embodiment is mainly described.

The notification information generation unit 528 acquires the determination result by the change trend determination unit 533. The notification information generation unit 528 generates the notification information using the acquired determination result. The notification information generation unit 528 outputs the generated notification information to the transmission unit 529. The notification information output to the transmission unit 529 is transmitted to the walk measurement device 53 and output from the output device 55 connected to the walk measurement device 53. For example, the output device 55 performs notification by display information, voice information, vibration and the like that may be recognized by the user. The output device 55 may also be configured to notify the user of the determination result by the change trend determination unit 533 by the display information, voice information, or vibration.

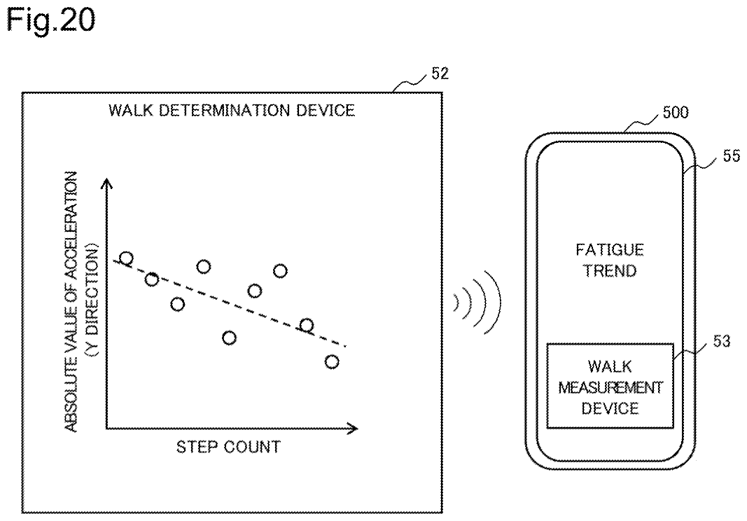

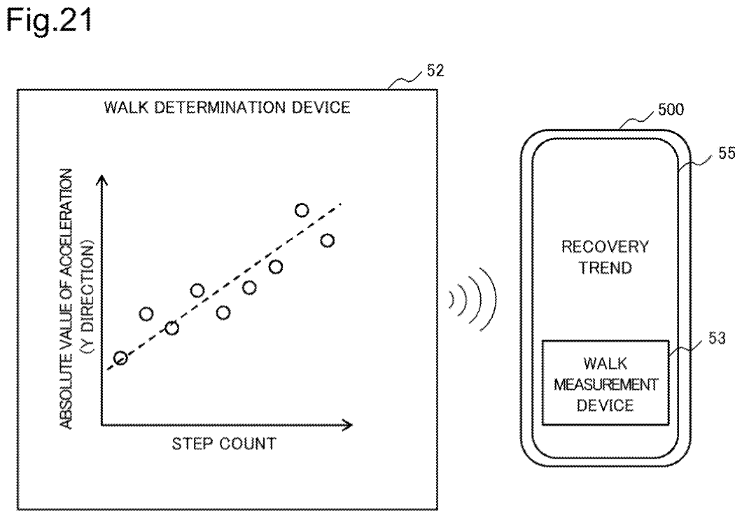

FIGS. 20 and 21 are conceptual diagrams illustrating an example in which the notification information according to the determination result by the change trend determination unit 533 is displayed on the output device 55. FIGS. 20 and 21 illustrate examples in which the walk measurement device 53 and the output device 55 are configured in a mobile terminal 500 such as a smartphone. FIGS. 20 and 21 are examples, and do not limit the notification information to be displayed on the output device 55.

FIG. 20 is an example of the notification information to be displayed on the output device 55 in a case where the change trend determination unit 533 determines that the change trend of the peak values is downward. In a case where the change trend of the peak values is downward, a walking speed of a user gradually decreases. In the example in FIG. 20, in a case where the change trend of the peak values is downward, notification information of “fatigue trend” is displayed on the output device 55.

FIG. 21 is an example of the notification information to be displayed on the output device 55 in a case where the change trend determination unit 533 determines that the change trend of the peak values is upward. In a case where the change trend of the peak values is upward, the walking speed of the user gradually increases. In the example in FIG. 21, in a case where the change trend of the peak values is upward, notification information of “recovery trend” is displayed on the output device 55.

The configuration of the walk measurement system of the present example embodiment is described above. The configuration in FIG. 19 is an example, and the configuration of the walk measurement system of the present example embodiment is not limited to this form.

(Operation)

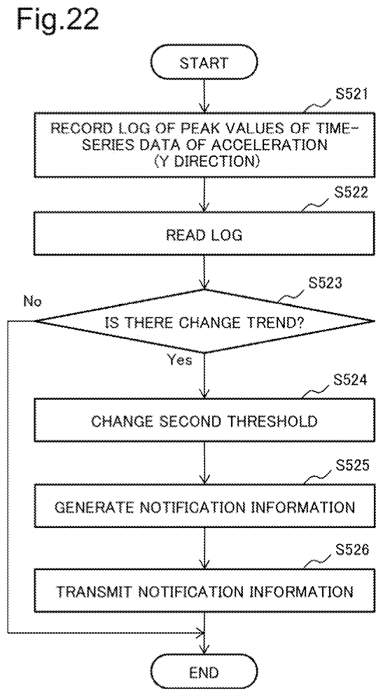

Next, an operation of the walk measurement system of the present example embodiment is described with reference to the drawings. Hereinafter, operation mode switching processing similar to that of the first example embodiment is omitted and determination processing of a walking trend (walking trend determination processing: FIG. 22) is described.

[Change Trend Determination Processing]

FIG. 22 is a flowchart for illustrating change trend determination processing executed when an operation mode is switched to a walk measurement mode. In the description along the flowchart in FIG. 22, the determination device 52 is described as a subject of the operation.

In FIG. 22, the determination device 52 first stores a log of the extreme values (peak values) of the time-series data of the acceleration in the travel direction (Y direction) (walking waveform) (step S521). For example, the determination device 52 at least stores the log used for determining the change trend of the peak values (for example, 10 steps).

Next, the determination device 52 reads the log used for determining the change trend of the peak values (step S522).

Here, the determination device 52 determines whether there is the change trend of the peak values (step S523). In a case where the change trend of the peak values is detected (Yes at step S523), the determination device 52 resets a second threshold based on the detected change trend (step S524). In contrast, in a case where the change trend is not detected (No at step S523), the processing along the flowchart in FIG. 22 ends.

After step S524, the determination device 52 generates the notification information according to the detected change trend (step S525). A timing of generating the notification information may be a timing parallel to step S524 or a timing before step S524.

Next, the determination device 52 transmits the generated notification information to the walk measurement device 53 (step S526). The notification information transmitted to the walk measurement device 53 is displayed on the output device 55.

The change trend determination processing by the determination device 52 of the present example embodiment is described above. The processing along the flowchart in FIG. 22 is an example, and the change trend determination processing by the determination device 52 is not limited to this procedure.

As described above, the walk measurement system of the present example embodiment is further provided with the output device connected to the walk measurement device and outputs the information received from the walk measurement device. The determination device is provided with the notification information generation unit that generates the notification information according to a detection result by the change trend detection unit. The transmission unit transmits the notification information generated by the notification information generation unit to the walk measurement device. The walk measurement device receives the notification information transmitted from the transmission unit, and transmits the received notification information to the output device. The output device receives the notification information transmitted from the walk measurement device, and outputs the received notification information.

According to the walk measurement system of the present example embodiment, it is possible to notify the user of a change in walking state by outputting the notification information according to the detection result by the change trend detection unit from the output device.

(Hardware)

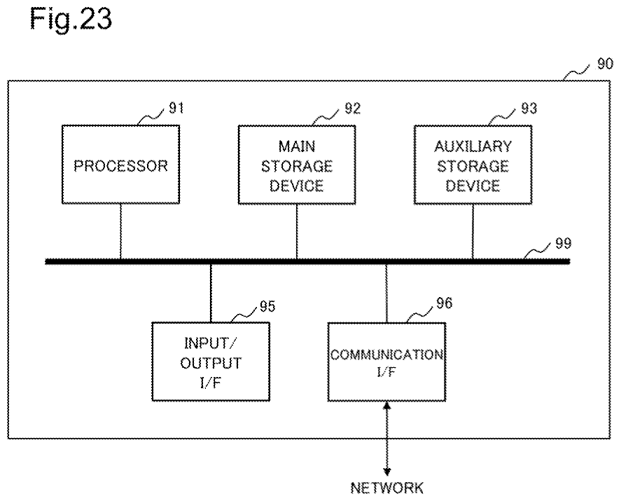

Here, a hardware configuration for executing the processing of the walk measurement system according to each example embodiment of the present invention is described using an information processing device 90 in FIG. 23 as an example. The information processing device 90 in FIG. 23 is a configuration example for executing the processing of the walk measurement system of each example embodiment, and does not limit the scope of the present invention.

As illustrated in FIG. 23, the information processing device 90 is provided with a processor 91, a main storage device 92, an auxiliary storage device 93, an input/output interface 95, and a communication interface 96. In FIG. 23, the interface is abbreviated as I/F. The processor 91, the main storage device 92, the auxiliary storage device 93, the input/output interface 95, and the communication interface 96 are data-communicably connected to each other via a bus 99. The processor 91, the main storage device 92, the auxiliary storage device 93, and the input/output interface 95 are connected to a network such as the Internet or an intranet via the communication interface 96.

The processor 91 develops a program stored in the auxiliary storage device 93 and the like in the main storage device 92 and executes the developed program. In the present example embodiment, a software program installed in the information processing device 90 may be used. The processor 91 executes processing by the walk measurement system according to the present example embodiment.

The main storage device 92 includes an area in which the program is developed. The main storage device 92 may be a volatile memory such as a dynamic random access memory (DRAM). A nonvolatile memory such as a magnetoresistive random access memory (MRAM) may be configured and added as the main storage device 92.

The auxiliary storage device 93 stores various data. The auxiliary storage device 93 is formed of a local disk such as a hard disk or a flash memory. Various data may be stored in the main storage device 92, and the auxiliary storage device 93 may be omitted.

The input/output interface 95 is an interface for connecting the information processing device 90 to a peripheral device. The communication interface 96 is an interface for connecting to an external system or device through a network such as the Internet or an intranet based on standards or specifications. The input/output interface 95 and the communication interface 96 may be made a shared interface to be connected to an external device.

An input device such as a keyboard, a mouse, or a touch panel may be connected to the information processing device 90 as necessary. These input devices are used to input information and settings. In a case where the touch panel is used as the input device, a display screen of the display device may also serve as an interface of the input device. Data communication between the processor 91 and the input device may be mediated by the input/output interface 95.

The information processing device 90 may be provided with a display device for displaying information. In a case where the display device is provided, the information processing device 90 is preferably provided with a display control device (not illustrated) for controlling the display of the display device. The display device may be connected to the information processing device 90 via the input/output interface 95.

The information processing device 90 may be provided with a disk drive as necessary. The disk drive is connected to the bus 99. The disk drive mediates reading of data and program from a recording medium, writing of a processing result of the information processing device 90 to the recording medium and the like between the processor 91 and the recording medium not illustrated (program recording medium). The recording medium may be achieved by, for example, an optical recording medium such as a compact disc (CD) or a digital versatile disc (DVD). The recording medium may be achieved by a semiconductor recording medium such as a universal serial bus (USB) memory or a secure digital (SD) card, a magnetic recording medium such as a flexible disk, or another recording medium.

The above is an example of the hardware configuration for enabling the walk measurement system according to each example embodiment of the present invention. The hardware configuration in FIG. 23 is an example of a hardware configuration for executing arithmetic processing of the walk measurement system of each example embodiment, and does not limit the scope of the present invention. A program for causing a computer to execute processing regarding the walk measurement system according to each example embodiment is also included in the scope of the present invention. A program recording medium in which the program according to each example embodiment is recorded is also included in the scope of the present invention.

The components of the walk measurement system of each example embodiment may be optionally combined. The components of the walk measurement system of each example embodiment may be achieved by software or may be achieved by a circuit.

While the present invention has been particularly shown and described with reference to example embodiments thereof, the present invention is not limited to these example embodiments. It will be understood by those of ordinary skill in the art that various changes in form and details may be made therein without departing from the spirit and scope of the present invention as defined by the claims.

Some or all of the above-described example embodiments may be described as in the following supplementary notes, but are not limited to the following.

(Supplementary Note 1)

A determination device provided with:

a sensor data reception unit that receives sensor data including acceleration in a travel direction of a user and in a gravity direction;

a walking state discrimination unit that switches to a discrimination mode in response to the acceleration in the gravity direction exceeding a first threshold in a power saving mode, and switches to a walk measurement mode in response to the acceleration in the travel direction exceeding a second threshold in the discrimination mode;

a change trend detection unit that detects a change trend of peak values using log data of the peak values of the acceleration in the travel direction in the discrimination mode;

a threshold change unit that changes the second threshold based on a detection result by the change trend detection unit; and

a transmission unit that transmits the sensor data in the walk measurement mode.

(Supplementary Note 2)

The determination device according to supplementary note 1, in which

the change trend detection unit

detects the change trend of the peak values using the log data of the peak values associated to heel contact of the user.

(Supplementary Note 3)

The determination device according to supplementary note 1 or 2, in which

the change trend detection unit includes:

a log storage unit that stores the log data of the peak values of the acceleration in the travel direction in the discrimination mode;

a log reading unit that reads the log data of the peak values stored in the log storage unit; and

a change trend determination unit that determines the change trend of the peak values using the log data of the peak values.

(Supplementary Note 4)

The determination device according to any one of supplementary notes 1 to 3, in which

the change trend detection unit

determines that the change trend of the peak values is upward in a case where a slope of a regression line obtained by performing linear regression on the log data of the peak values is positive, and determines that the change trend of the peak values is downward in a case where the slope of the regression line is negative, and

the threshold change unit

increases an absolute value of the second threshold in a case where the change trend of the peak values is determined to be upward, and decreases the absolute value of the second threshold in a case where the change trend of the peak values is determined to be downward.

(Supplementary Note 5)

The determination device according to supplementary note 4, in which

the change trend detection unit

includes a determination criterion storage unit for storing a determination criterion regarding a range of the slope of the regression line within which the change trend of the peak values is determined to be constant, and

determines that the change trend of the peak values is constant in a case where the slope of the regression line is within the range of the determination criterion, and

the threshold change unit

does not change the second threshold in a case where the change to trend of the peak values is determined to be constant.

(Supplementary Note 6)

The determination device according to any one of supplementary notes 1 to 5, provided with:

a learning model storage unit that stores a learning model regarding the change trend of the peak values, in which

the change trend detection unit

detects the change trend of the peak values by applying the log data of the peak values to the learning model.

(Supplementary Note 7)

The determination device according to any one of supplementary notes 1 to 6, in which

the threshold change unit includes:

a threshold storage unit that stores the first threshold set for the acceleration in the gravity direction and the second threshold set for the acceleration in the travel direction; and

a threshold setting unit that sets the second threshold stored in the threshold storage unit based on a determination result by the change trend detection unit.

(Supplementary Note 8)

A walk measurement system provided with:

the determination device according to any one of supplementary notes 1 to 7; and

a data acquisition device arranged in footwear of the user, the data acquisition device that generates the sensor data according to an operation mode switched by the walking state discrimination unit, and transmits the generated sensor data to the determination device.

(Supplementary Note 9)

The walk measurement system according to supplementary note 8, provided with:

a walk measurement device that receives the sensor data transmitted from the transmission unit, and measures a walk of the user using the received sensor data.

(Supplementary Note 10)

The walk measurement system according to supplementary note 9, further provided with:

an output device connected to the walk measurement device, the output device that outputs information received from the walk measurement device, in which

the determination device

includes a notification information generation unit that generates notification information according to a determination result by the change trend detection unit,

the transmission unit

transmits the notification information generated by the notification information generation unit to the walk measurement device,

the walk measurement device

receives the notification information transmitted from the transmission unit, and transmits the received notification information to the output device, and

the output device

receives the notification information transmitted from the walk measurement device, and outputs the received notification information.

(Supplementary Note 11)

A determination method provided with:

receiving sensor data including acceleration in a travel direction of a user and in a gravity direction;

switching to a discrimination mode in response to the acceleration in the gravity direction exceeding a first threshold in a power saving mode;

switching to a walk measurement mode in response to the acceleration in the travel direction exceeding a second threshold in the discrimination mode;

transmitting the sensor data in the walk measurement mode;

detecting a change trend of peak values using log data of the peak values of the acceleration in the travel direction in the discrimination mode; and

changing the second threshold based on the change trend of the peak values.

(Supplementary Note 12)

A program for causing a computer to execute:

processing for receiving sensor data including acceleration in a travel direction of a user and in a gravity direction;

processing for switching to a discrimination mode in response to the acceleration in the gravity direction exceeding a first threshold in a power saving mode;

processing for switching to a walk measurement mode in response to the acceleration in the travel direction exceeding a second threshold in the discrimination mode;

processing for transmitting the sensor data in the walk measurement mode;

processing for detecting a change trend of peak values using log data of the peak values of the acceleration in the travel direction in the discrimination mode; and

processing for changing the second threshold based on the change trend of the peak values.

### REFERENCE SIGNS LIST

1 walk measurement system
10 walk determination device
11, 21, 31, 41, 51 data acquisition device
12, 22, 32, 42, 52 determination device
13, 23, 33, 43, 53 walk measurement device
55 output device
110 sensor
111 acceleration sensor
112 angular velocity sensor
113 signal processing unit
114 data transmission unit
121, 221, 321, 421, 521 sensor data reception unit
122, 222, 322, 422, 522 walking state discrimination unit
123, 223, 323, 423, 523 change trend detection unit
125, 225, 325, 425, 525 threshold change unit
129, 229, 329, 429, 529 transmission unit
131, 231, 331, 431, 531 log storage unit
132, 232, 332, 432, 532 log reading unit
133, 233, 333, 433, 533 change trend determination unit
151, 251, 351, 451, 551 threshold storage unit
152, 252, 352, 452, 552 threshold setting unit
335 determination criterion storage unit
427 learning model storage unit
528 notification information generation unit

What is claimed is:

1. A determination device comprising:
at least one memory storing instructions; and
at least one processor connected to the at least one memory and configured to execute the instructions to:
receive sensor data including acceleration in a travel direction of a user and in a gravity direction;
switch to a discrimination mode in response to the acceleration in the gravity direction exceeding a first threshold in a power saving mode,
switch to a walk measurement mode in response to the acceleration in the travel direction exceeding a second threshold in the discrimination mode;
detect a change trend of peak values using log data of the peak values of the acceleration in the travel direction in the discrimination mode;
change the second threshold based on a detection result of the change trend of the peak value; and
transmit the sensor data in the walk measurement mode.

2. The determination device according to claim 1, wherein
the at least one processor is configured to execute the instructions to
detect the change trend of the peak values using the log data of the peak values associated to heel contact of the user.

3. The determination device according to claim 1, wherein
the at least one processor is configured to execute the instructions to:
store the log data of the peak values of the acceleration in the travel direction in the discrimination mode;
read the stored log data of the peak values; and
determine the change trend of the peak values using the log data of the peak values.

4. The determination device according to claim 1, wherein
the at least one processor is configured to execute the instructions to
determine that the change trend of the peak values is upward in a case where a slope of a regression line obtained by performing linear regression on the log data of the peak values is positive,
determine that the change trend of the peak values is downward in a case where the slope of the regression line is negative,
increase an absolute value of the second threshold in a case where the change trend of the peak values is determined to be upward, and
decrease the absolute value of the second threshold in a case where the change trend of the peak values is determined to be downward.

5. The determination device according to claim 4, wherein
the at least one processor is configured to execute the instructions to
store a determination criterion regarding a range of the slope of the regression line within which the change trend of the peak values is determined to be constant,
determine that the change trend of the peak values is constant in a case where the slope of the regression line is within the range of the determination criterion, and
do not change the second threshold in a case where the change trend of the peak values is determined to be constant.

6. The determination device according to claim 1, wherein
the at least one processor is configured to execute the instructions to
store a learning model regarding the change trend of the peak values, and
detect the change trend of the peak values by applying the log data of the peak values to the learning model.

7. The determination device according to claim 1, wherein
the at least one processor is configured to execute the instructions to
store the first threshold set for the acceleration in the gravity direction and the second threshold set for the acceleration in the travel direction; and
set the stored second threshold based on the detection result of the change trend of the peak value.

8. A walk measurement system comprising:
the determination device according to claim 1; and
a data acquisition device that is arranged in footwear of the user, generates the sensor data according to an operation mode switched by the determination device, and transmits the generated sensor data to the determination device.

9. The walk measurement system according to claim 8, comprising:
a walk measurement device that receives the sensor data transmitted from the determination device, and measures a walk of the user using the received sensor data.

10. The walk measurement system according to claim 9, further comprising:
an output device that is connected to the walk measurement device, and outputs information received from the walk measurement device, wherein
the at least one processor of the determination device is configured to execute the instructions to
generate notification information according to a detection result of change trend of a peak value of an acceleration in a travel direction in a discrimination mode,
transmit the generated notification information to the walk measurement device,
the walk measurement device is configured to
receive the notification information transmitted from the determination device, and transmit the received notification information to the output device, and
the output device is configured to
receive the notification information transmitted from the walk measurement device, and output the received notification information.

11. A determination method comprising:
receiving sensor data including acceleration in a travel direction of a user and in a gravity direction;
switching to a discrimination mode in response to the acceleration in the gravity direction exceeding a first threshold in a power saving mode;
switching to a walk measurement mode in response to the acceleration in the travel direction exceeding a second threshold in the discrimination mode;
transmitting the sensor data in the walk measurement mode;
detecting a change trend of peak values using log data of the peak values of the acceleration in the travel direction in the discrimination mode; and
changing the second threshold based on the change trend of the peak values.

12. A non-transient program recording medium recording a program for causing a computer to execute:
processing for receiving sensor data including acceleration in a travel direction of a user and in a gravity direction;
processing for switching to a discrimination mode in response to the acceleration in the gravity direction exceeding a first threshold in a power saving mode;
processing for switching to a walk measurement mode in response to the acceleration in the travel direction exceeding a second threshold in the discrimination mode;
processing for transmitting the sensor data in the walk measurement mode;
processing for detecting a change trend of peak values using log data of the peak values of the acceleration in the travel direction in the discrimination mode; and
processing for changing the second threshold based on the change trend of the peak values.
